(19) United States
(12) Patent Application Publication
(10) Pub. No.: US 2024/0399175 A1
(43) Pub. Date: Dec. 5, 2024

(54) REVERSAL OF SENESCENCE BY ULTRASOUND IRRADIATION

(71) Applicant: Board of Regents, The University of Texas System, Austin, TX (US)

(72) Inventors: Michael Sheetz, Galveston, TX (US); Sanjay Kureel, Galveston, TX (US); Blake Rasmussen, Galveston, TX (US); Rosario Maroto, Galveston, TX (US)

(21) Appl. No.: 18/719,116

(22) PCT Filed: Dec. 1, 2022
(86) PCT No.: PCT/US2022/051482

§ 371 Date: Jun. 12, 2024

Related U.S. Application Data
(60) Provisional application No. 63/290,969, filed on Dec. 17, 2021.

(51) Int. Cl. A61N 7/02; A61N 7/00

(52) U.S. Cl. CPC A61N 7/02; A61N 2007/0017; A61N 2007/0034; A61N 2007/0078

(57) ABSTRACT

The present invention includes non-invasive methods of treating aging using low frequency structured and modulated ultrasound. The ultrasound irradiation causes mechanical stretching of cells even within the body of an organism which in turn can reverse the characteristics of senescent cells to counteract the effect of senescent cells on the function of the tissues wherein they reside. Senescence reversal by ultrasound involves at least one of: activates cell growth, reduces cell size, increases secretion of growth factors, increases mitochondrial fission and/or promotes wound healing. Ultrasound treatment improves the function of aged individuals, of specific organs, of wound healing and enables the greater expansion of normal cells in vitro. Ultrasound can be delivered in spas for the whole organism or smaller targeted ultrasound devices can be made for specific organs or cell applications.

### TECHNICAL FIELD OF THE INVENTION

The present invention relates in general to the field of aging, and more particularly, to the reversal of senescence by ultrasound irradiation.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with aging.

As the average lifespan of people increases and the population gets older, there are more and more people who suffer from age-related disorders that generally impair their function and quality of life. Many of the problems associated with aging can be explained as the result of cellular senescence. Senescent cells do not grow or function properly and secrete factors that increase senescence of neighboring cells.

What are needed are treatments for aged adults that decrease the fraction of senescent cells in the body, thus, the adverse effects of aging will be decreased, and the quality of life will be improved in old age. Although there are a number of factors that can increase the lifespan, they have generally not been analyzed for effects on quality of life.

There are currently two major approaches to combating aging that appear to increase quality of life, exercise and senolytics. Continued exercise in aged adults has many benefits and helps to diminish aging effects on physical as well as mental performance. Senolytics are drugs or small molecules that cause the apoptosis of senescent cells and have been shown to improve performance of aged mice by decreasing the fraction of senescent cells in the body. Since some consider exercise a senolytic, the two approaches may act through a common target, i.e., diminishing the fraction of senescent cells. In terms of weaknesses of these approaches, exercise is difficult to sustain because of injuries, lifestyle issues, and personal will. Unfortunately, senolytics are difficult to target to senescent cells only and often have side effects.

Despite these advances, what are needed are novel treatment methodologies that help reduce the rate of aging, reverse cellular senescence, and/or help reduce or eliminate agents released by senescent cells that increase the rate of aging.

### SUMMARY OF THE INVENTION

The present invention mimics the mechanical effects of exercise on senescent cells using ultrasound irradiation. It is demonstrated herein that the mechanical effects of ultrasound reverse cell senescence without killing the cells. Additional benefits of low frequency ultrasound (US) are that it can penetrate the whole body to act on internal senescent cells and reverse senescence in situ to enable the cells to return to a functional state.

As embodied and broadly described herein, an aspect of the present disclosure relates to a non-invasive method of treating aging comprising: mechanically stretching at least one living cell in an amount sufficient to delay at least one aging characteristic. In one aspect, the at least one aging characteristic is selected from reduced cellular senescence, increased cell division, reduced cell size, increase secretion of growth factors, decreased secretion of senescent factors, reduced mitochondrial fusion, increased mitochondrial fission, or enhanced wound healing. In another aspect, mechanically stretching at least one living cell is by applying a repetitive low frequency ultrasound that is configured to target a region in need of treatment for aging, wherein the region is targeted by one or more ultrasound sources pointed at the target from one or more directions. In another aspect, an immersion design for the treatment is optimized to treat wounds and ulcers and can include one or more chambers that are generally shallow and suited for full immersion with an absorber shield on the far side of the treatment zone, and wherein the transducers and an absorber can contact a patient or are insulated by the immersion medium. In another aspect, mechanically stretching at least one living cell is by applying the repetitive low frequency ultrasound treatment is selected from at least one of: standing wave patterns, shock waves, rapid sounds transitions, square waves, sawtooth waves, random waves, or undulating a low-high intensity beat. In another aspect, mechanically stretching the at least one living cell is by applying the repetitive low frequency ultrasound treatment of between about 5 minutes to about 60 minutes in duration and is repeated for a period selected from the group consisting of: once a day, about once every two days, about once every three days, about once a week, and about twice a week. In another aspect, mechanically stretching at least one living cell is by applying the repetitive low frequency ultrasound treatment is for 1, 2, 3, 4, 5, 6, 7, 8, 9, 10, 15, 20, 25, 30, 40, 50, 60, 70, 75, 80, 90 minutes, or 2, 3, 4, 5, 6, 7, 8, 9, 10, 11, 12, or 24 hours. In another aspect, mechanically stretching at least one living cell is by applying the repetitive, usually low frequency ultrasound treatment is at 5, 10, 15, 20, 25, 30, 35, 40, 45, 50, 60, 75, 100, 150, 200, 250, 300, 400, 500, 600, 700, 750, 800, 900 kHz, 1 MHz or higher. In another aspect, mechanically stretching at least one living cell is by applying the repetitive low frequency ultrasound treatment is at 10, 20, 50, 100, 150, 200, 250, 300, 350, 400, 450, 500, 1,000, 5,000, 10,000, or <500 mW/cm². In another aspect, at least one living cell is a cell line or cell clone (or other primary cells), or in a tissue, organ, limb, or whole body. In another aspect, at least a portion of mechanically stretching at least one living cell by a repetitive low frequency ultrasound treatment applied concurrently with one or more active agents that reduce cellular senescence. In another aspect, the active agent is a supernatant of cells treated with a repetitive low frequency ultrasound treatment. In another aspect, mechanically stretching at least one living cell is a repetitive low frequency ultrasound treatment delivered from at least one of: one or more ultrasound transducers configured to generate a sequence of programmed cycles of waves; one or more ultrasound transducers mounted to a robotic arm, wherein the robotic arm is controlled to position the one or more ultrasound transducers into a desired location or orientation; one or more ultrasound transducers are phased array ultrasound transducers; or the one or more ultrasound transducers are sealed and water-proof; wherein a frequency, magnitude of the periodic force and the period of time are determined based at least in part on a type of the target cells or based on the output of a feedback sensor at or near a treatment zone; or wherein a wavelengths of a wave is in the order of a size of an organ and an amplitude in the order of single cells. In another aspect, mechanically stretching at least one living cell is a sequence of programmed cycles of waves generated according to a treatment plan or a type of therapeutic procedure, a treatment plan is generated using one or more machine learning techniques, a lower frequency modulation of a sound wave, a cyclical on-off pattern with a set duty-cycle, a variation of amplitude, frequency, and phase; a superposition of several carrier frequencies each variable in phase and center frequency and relative intensity; modulated by an external input signal, modulated by an audible sound signal. In another aspect, the method further comprising providing a controller, wherein the controller is: configured to control one or more ultrasound transducers based on sensor data; integrated with one or more ultrasound generators; comprises remote drivers that limit the energy from the one or more ultrasound generators to provide patient safety; neutral drivers with a voltage across two or more ultrasound transducer elements that amounts to zero at all times; or connected to one or more transducer stacks that reduces a peak voltage used by one or more ultrasound generators. In another aspect, at least one living cell is a plurality of living cells, and the method further comprises applying repetitive low frequency ultrasound treatment to a plurality of in vivo living cells. In another aspect, mechanically stretching at least one living cell is a repetitive low frequency ultrasound treatment delivered to at least a localized region of the body of a subject. In another aspect, the repetitive low frequency ultrasound treatment is provided by a piezo transducer, a voice coil, a capacitive membrane, fluidic instability, a shuttered hydraulic, a pneumatic device, a spark discharge, chemically generated pressure waves, engine driven sound, induced muscle tonus, a transducer array, a phased array with a synthetic aperture, or harmonics of the frequencies. In another aspect, the at least one living cell is a plurality of living cells and the applying repetitive low frequency ultrasound treatment extends the replicative lifespan of the at least one living cell. In another aspect, at least one living cell is a plurality of living cells and the applying repetitive low frequency ultrasound treatment reverts the plurality of living cells to a more youthful phenotype. In another aspect, the method further comprises providing a mechanism that at least one of: absorbs or uncouples the repetitive low frequency ultrasound treatment to reduce or deflect a sound wave after patient treatment, is a material that is suitable to at least one of attenuate the sound waves or guide the sound waves away from a site or treatment, or includes one or more elements that convert residual sound energy into heat.

As embodied and broadly described herein, an aspect of the present disclosure relates to a method of reducing cellular senescence comprising: applying a repetitive low frequency ultrasound treatment to at least one living cell, wherein the repetitive low frequency ultrasound treatment to delay at least one aging characteristic. In one aspect, the wavelengths are equal to or greater than an average cell diameter. In another aspect, the at least one aging characteristic is selected from reduced cellular senescence, increased cell division, reduced cell size, increase secretion of growth factors, decreases secretion of senescent factors, prevents mitochondrial fusion, increases mitochondrial fission, or enhances wound healing. In another aspect, applying the repetitive low frequency ultrasound is configured to target a region in need of treatment for aging, wherein the region is targeted by one or more ultrasound sources pointed at the target from one or more directions. In another aspect, applying the repetitive low frequency ultrasound treatment is selected from at least one of: standing wave patterns, shock waves, rapid transitions, square waves, sawtooth waves, random waves, or undulating a low-high intensity beat. In another aspect, applying the repetitive low frequency ultrasound treatment is between about 5 minutes to about 30 minutes in duration and is repeated for a period selected from the group consisting of: once a day, about once every two days, about once every three days, about once a week, and about twice a week. In another aspect, applying the repetitive low frequency ultrasound treatment is for 1, 2, 3, 4, 5, 6, 7, 8, 9, 10, 15, 20, 25, 30, 40, 50, 60, 70, 75, 80, 90 minutes, or 2, 3, 4, 5, 6, 7, 8, 9, 10, 11, 12, or 24 hours. In another aspect, applying the repetitive low frequency ultrasound treatment is at 5, 10, 15, 20, 25, 30, 35, 40, 45, 50, 60, 75, 100, 150, 200, 250, 300, 400, 500, 600, 700, 750, 800, 900 kHz, 1 MHz or higher. In another aspect, applying the repetitive low frequency ultrasound treatment is at 10, 20, 50, 100, 150, 200, 250, 300, 350, 400, 450, 500, or <500 mW/cm². In another aspect, at least one living cell is a cell line or cell clone (such as a primary cell), or in a tissue, organ, limb, or whole body. In another aspect, at least a portion of the application of the repetitive low frequency ultrasound treatment is applied concurrently with an active agent the reduces cellular senescence. In another aspect, the active agent is a supernatant of cells treated with the repetitive low frequency ultrasound treatment. In another aspect, the repetitive low frequency ultrasound treatment is applied to the at least one living cell until the cell reaches cellular senescence to delay at least one aging characteristic. In another aspect, the repetitive low frequency ultrasound treatment is delivered from at least one of: one or more ultrasound transducers configured to generate a sequence of programmed cycles of waves; one or more ultrasound transducers mounted to a robotic arm, wherein the robotic arm is controlled to position the one or more ultrasound transducers into a desired location or orientation; one or more ultrasound transducers are phased array ultrasound transducers; or the one or more ultrasound transducers are sealed and water-proof; wherein a frequency, magnitude of the periodic force and the period of time are determined based at least in part on a type of the target cells or based on the output of a feedback sensor at or near a treatment zone; or wherein a wavelengths of a wave is in the order of a size of an organ and an amplitude in the order of single cells. In another aspect, the repetitive low frequency ultrasound treatment is at least one of: a sequence of programmed cycles of waves generated according to a treatment plan or a type of therapeutic procedure; a treatment plan is generated using one or more machine learning techniques; a lower frequency modulation of a sound wave, a cyclical on-off pattern with a set duty-cycle; a variation of amplitude, frequency, and phase; a superposition of several carrier frequencies each variable in phase, center frequencies and relative intensities; a signal modulated by an external input signal, or a signal modulated by an audible sound. In another aspect, the method further comprises providing a controller, wherein the controller is: configured to control the one or more ultrasound transducers based on sensor data; integrated with one or more ultrasound generators; comprises remote drivers that limit the energy from the one or more ultrasound generators to provide patient safety; neutral drivers with a voltage across one or more ultrasound transducer elements that sums up to zero at all times; or connected to one or more transducer stacks that reduces a peak voltage used by the one or more ultrasound generators. In another aspect, the at least one living cell is a plurality of living cells, and the method further comprises applying repetitive low frequency ultrasound treatment to a plurality of in vivo living cells. In another aspect, the repetitive low frequency ultrasound treatment is delivered to at least a localized region of the body of a patient. In another aspect, the repetitive low frequency ultrasound treatment is provided by a piezo transducer, a voice coil, a capacitive membrane, fluidic instability, a shuttered hydraulic, a pneumatic device, a spark discharge, chemically generated pressure waves, engine driven sound, induced muscle tonus, a phased array, a phased array with one or more synthetic apertures, or harmonics. In another aspect, at least one living cell is a plurality of living cells and applying the repetitive low frequency ultrasound treatment extends the replicative lifespan of at least one living cell. In another aspect, at least one living cell is a plurality of living cells and the applying repetitive low frequency ultrasound treatment reverts the plurality of living cells to a more youthful phenotype. In another aspect, the method further comprises providing a mechanism that at least one of: absorbs or uncouples the repetitive low frequency ultrasound treatment to reduce or deflect a sound wave after patient treatment, is a material that is suitable to at least one of attenuate the sound waves or guide the sound waves away from a site or treatment, or includes one or more elements that convert residual sound energy into heat. In another aspect, the method further comprises providing an immersion vessel to treat wounds and ulcers that includes one or more chambers that are partial or full immersion with an absorber shield about a treatment zone, and wherein one or more transducers and one or more absorber can contact a patient or are insulated by the immersion medium.

### BRIEF DESCRIPTION OF THE DRAWINGS

For a more complete understanding of the features and advantages of the present invention, reference is now made to the detailed description of the invention along with the accompanying figures and in which:

FIGS. 1A to 1H show the characterization of senescent cells. (FIG. 1A) Brightfield images of senescent cells induced by H₂O₂, sodium butyrate (SB), Doxorubicin (Dox), and Bleomycin Sulphate (BS). Scale bar=300 μm. (FIG. 1B) Quantification of proliferation shows no senescent growth after a 48 h incubation. (FIG. 1C) Senescent cells become enlarged compared to the normal control cells. (FIG. 1D) Quantification of avg. cell volume of bleomycin sulfate treated cells compared to control cells. (FIG. 1E) Conditioned medium from senescent cells inhibits the growth of normal proliferating cells. (FIG. 1F) SA-b-galactosidase staining of control (proliferating) and BS treated senescent cells. Scale bar=300 μm. (FIG. 1G) Level of b-galactosidase senescence marker in H₂O₂ and SB induced SCs. Scale bar=300 μm. Data in (FIG. 1B)-(1G) Minimum of 150 cells were analyzed for spread area and cell volume from n=2 experiments.

FIGS. 2A to 2G show that ultrasound reverses cell senescence. (FIG. 2A) Schematic illustration of senescence reversal experiment. SB treated Vero cells were treated with low frequency ultrasound (LFU) and passaged after every 48 h for 8-10 days. (FIG. 2B) Graph showing growth of cells as fold change over 48 hr. treated cells passage from P0 to P3 every 48 h. (FIG. 2C) Cell area of LFU treated senescent cells (LFU) is largely restored to normal by P3. (FIG. 2D) Representative anti-p21 immunofluorescence images of P3 control and LFU treated cells. Scale bar=300 μm. (FIG. 2E) Quantification of fluorescence intensity of p21 stained control and LFU treated P3 cells. Shown as mean±SD for >200 cells in each condition. (FIG. 2F) Ultrasound increased proliferation as determined by EDU staining. (FIG. 2G) Quantification of EDU positive P3 cells. All graphs were plotted by mean±SD. Minimally 200 cells were analyzed in each condition.

FIGS. 3A to 3H show that senescence associated secretory phonotype (SASP) inhibits control, but USS increases senescent cell growth. (FIG. 3A) Schematic illustration of the experiment. SCs were cultured in growth medium for 24 hours then they were treated with LFU for 30 min. Supernatant was collected after the LFU treatment (S0) and again supernatant was collected after 24 h (S24). To check the effect of LFU treatment, supernatants S0 and S24 were used to check the growth of non-senescent control cells. (FIG. 3B) Representative brightfield images of control cells after 48 hours of incubation in normal growth medium, S0, and S24. (FIGS. 3C and 3D) Quantification of control cell numbers shows growth and normal size in control and S24 media but reduced growth and increased size in S0. (FIG. 3E) Timeline and strategy of schematic of LFU treatment of normal proliferating cells. Schematic showing that control cells were treated with US four times in the same media and the supernatant was collected (USS) for incubation of senescent cells for 48 hours. (FIG. 3F) Brightfield images shows change in morphology of SCs in USS collected from LFU treated control cells. Senescent cells in normal growth medium were the controls. (FIG. 3G) Graph shows increased growth of SCs in USS and (FIG. 3H) decreased spread area in USS of senescent cells. Graphs were plotted by mean SD. Minimally 200 cells were analyzed for graphs (FIG. 3C) and (FIG. 3H). Scale bar=300 μm.

FIGS. 4A to 4D show that low frequency ultrasound decreases mitochondrial length and lysosome intensity in senescent cells. (FIG. 4A) Representative immunofluorescent images of mitochondrial morphology and Lysosome fluorescence in normal, senescent and LFU-treated senescent cells stained with Mitotracker and lysosome tracker. Scale bar=10 μm. (FIG. 4B) Ratio of intensities of lysosomal to mitochondrial staining is decreased by ultrasound treatment of senescent cells. (FIG. 4C) Quantification of mitochondrial length shows decreased length after LFU. Results are shown as mean±SD, minimum 8 cells were analyzed, n>3 experiments and significance was determined using two-tailed unpaired t-test. ***p value<0.001. (FIG. 4D) Diagram of a working model for the rejuvenation of senescent cells by activation of autophagy through LFU inhibition of mTORC1 activity.

FIGS. 5A to SE show that ultrasound reversal of replicative senescence enables growth of a larger number of cells. (FIG. 5A) Growth rate calculated as numbers of cumulative population doublings (CPD) for Control HFF and LFU treated HFF cells passaged every 48 hours from P13 to P24 passage and treated every other passage. (FIG. 5B). LFU treated cells were smaller than the p24 control and even p13 cells. (FIG. 5C) Fraction of SA-β-galactosidase positive cells decreased after LFU treatment. (FIG. 5D) Similarly LFU treatment of MSCs expanded the cell number P10-P19, with treatment every other passage. (FIG. 5E) LFU treated MSCs showed normal differentiation to (ORO) adipocytes and (ARS) osteocytes. Alizarin red S dye marked osteogenesis (ARS) and oil red O dye marked lipid droplets (ORO). Results are shown as mean±SD, minimally 200 cells for spread area and 150 cells for percentage β-galactosidase analysis, n>3 experiments, and significance was determined using two tailed unpaired t-test. ***p value<0.001, **p value<0.01, and *p value<0.05.

FIG. 6 is a side view of a LFU device showing the treatment of a foot using the present invention.

FIGS. 7A to 7E show the effects of ultrasound treatment on performance of aged mice. (FIG. 7A) Schematic illustration of the treatment plan. Mice (22-24 month old C57BL/6J strain) were LFU treated for 30 min every 3^{rd} day and/or ran on the treadmill 12 times for 20 min over one month. They then had a month break and were treated or exercised for a second month. Each study group contained four male and four female mice. (FIG. 7B) Graph of results of inverted cling test after the first month. and (FIG. 7C) after the second month of treatment. (FIG. 7D) Results of the treadmill test after the first month and (FIG. 7E) after the second month of treatment. Groups were untreated (Sham), treated with LFU (US), treated with exercise (EX), treated with rapamycin (RAP), treated with LFU plus exercise (EXUS), and treated with rapamycin plus LFU (RUS). Results are plotted as mean±S.D. Student t-test was used to determine the statistical significance. P-value greater than 0.05 is represented by ns. Statistical significance was given in p-value. *p value<0.05, **p value<0.001, and ***p value<0.0001.

FIGS. 8A to 8E show that LFU dramatically decreases the fraction of senescent cells in kidney and pancreas. 22-25 months old mice were treated with LFU every day with 1× power (LFU_D1), every other day (LFU_D2), every third day (LFU_D3) and every day with 1.3× power (LFU_1,3_D1). After two rounds of 2 weeks of treatment and assessment, mice were euthanized, and kidney and pancreas were collected for SA-β-galactosidase staining. (FIG. 8A) diagram of the experiment showing the sequence of events. (FIG. 8B) SA-β-galactosidase-stained kidney section of sham and LFU_D3 mice. In the color camera, β-galactosidase staining appeared blue and most of the cells in the kidney of sham mice were stained bot not in LFU_D3 kidney. (FIG. 8C) SA-β-galactosidase-stained images of pancreas section shown in color. Scale bar=150 μm. and 20 μm. (FIG. 8D) Quantification of β-galactosidase-stained area of kidney section. (FIG. 8D) Quantification of beta staining of pancreas section. β-galactosidase staining was reduced significantly in all LFU treated mice. Results are plotted as mean±S.D. Student t-test was used to determine the statistical significance. P-value greater than 0.05 is represented by ns. Statistical significance was given in p-value. *p value<0.05, **p value<0.001, and ***p value<0.0001 (n=10 mice).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Ultrasound rejuvenation is different from the existing therapies to combat aging such as exercise, senolytics, and other drug therapies in that it is non-invasive, has no known negative side effects, and can treat the whole organism and even internal organs. There is evidence that exercise and ultrasound both act mechanically to reverse senescence through similar biochemical pathways. The advantage of LFU is that it can reach tissues and organs when the individual is unable to exercise them; thus, enabling rejuvenation of many more tissues. In the case of senolytic agents that cause selective apoptosis of senescent cells, they can reverse some aspects of aging in older mice^{5,6}. These are now under consideration for clinical trials to aid older individuals⁷. Because the senolytic agents involve biochemical agents, they are difficult to deliver locally and it is not clear that they will work on senescent cells from different tissues. Further, there are concerns about the duration of treatment of Senolytics^{8 9} and effector molecules may have adverse effects on other cell types simultaneously³. Also, the death of the senescent cells leaves gaps in the tissues that will need tissue growth to repair. It will be much better to rejuvenate senescent cells in situ to rapidly restore normal function. There are potentially other drugs or treatments that will enhance rejuvenation of senescent cells by enhancing the biochemical pathways involved. LFU can enhance those treatments since it can be administered flexibly to augment the changes needed. Importantly, mechanical treatments unlike drug or other therapies act through normal biochemical pathways and ultrasound provides a flexible way to activate those pathways non-invasively. Since ultrasound treatment reverses the senescent phenotype without cell death and can be administered repeatedly, LFU treatment provides a benefit over time for older individuals. LFU irradiation below the damage threshold—used as definition for “low power” here—also allows peak pressure of higher peak strain than readily achievable by exercise and acceleration forces several orders of magnitude higher than achievable with healthy exercise regimes. LFU irradiation can reach nearly all tissue types including those shielded by bones and bones themselves. The treatment regime of LFU is hence more universal than conventional exercise.

The present invention includes non-invasive methods of treating aging using low frequency ultrasound. The LFU irradiation causes mechanical stretching of cells even within the body of an organism and can reverse the characteristics of senescent cells to counteract the effect of senescent cells on the function of the tissues wherein they reside. Senescence reversal by LFU at least one of: activates cell growth, reduces cell size, increases secretion of growth factors, increases mitochondrial fission and/or promotes wound healing. LFU treatment improves the function of aged mice, of specific organs, of wound healing and enables the greater expansion of normal cells in vitro. Ultrasound can be delivered in spas for the whole organism or smaller targeted ultrasound devices can be made for specific organs or cell applications.

As used herein, the phrase “low frequency ultrasound (LFU)” refers to those wavelengths of the order of centimeters and amplitudes—the movement—smaller than several microns. In one example, the wavelengths are of the order of the size of organs and the amplitudes of the order of single cells. For example, repetitive low frequency ultrasound treatment can be at 5, 10, 15, 20, 25, 30, 35, 40, 45, 50, 60, 75, 100, 150, 200, 250, 300, 400, 500, 600, 700, 750, 800, 900 kHz, 1 MHz or higher, but often 5, 10, 15, 20, 25, 30, 35, 40, 45, 50, 60, 75, 100, 150, 200, 250, 300, 400, 500, 600, 700, 750, 800, 900 kHz, or 1 MHz. A repetitive low frequency ultrasound treatment can be at 10, 20, 50, 100, 150, 200, 250, 300, 350, 400, 450, 500, 1,000, 5,000, 10,000, but often <500 mW/cm².

As used herein, the term “treating” refers to inhibiting, preventing, curing, reversing, attenuating, alleviating, minimizing, suppressing, or halting the deleterious effects of a disease and/or causing the reduction, slowing, or regression of disease. Those of skill in the art will understand that various methodologies and assays can be used to assess the development of a disease, and similarly, various methodologies and assays may be used to assess the reduction, slowing or regression of aging.

Mechanotherapy. In certain aspects of the present disclosure, a mechanotherapy or ultrasound therapy is provided for generating and imparting periodic forces to senescent cells in a subject in need thereof. The immersion mechanotherapy may comprise applying one or more programmed cycles of waves to the subject in a uniform and immersion liquid for a pre-determined period of time. The programmed cycles of waves impart periodic forces with controlled duration, magnitude, and frequency that are sufficient to distort the target cells (e.g., senescent cells or tissues with senescent cells) such that a mechanically-induced rejuvenation process is triggered. generated periodic forces have low intensity and are small enough such that the normal cells and healthy tissues that the periodic forces impact on are not be subjected to mechanical or thermal damage. From a physical point of view, the periodic forces of ultrasound and exercise look remarkably different, especially as the force vectors during ultrasound treatment change tens of thousand times per second. The averaged stress and pressure impinged upon the tissue is, however, remarkably similar. When the cellular processes cannot follow the directional changes of the sound waves, the remaining force envelope corresponds to the intensity of the treatment.

In some cases, the frequency and magnitude of the periodic forces may be pre-determined based on the target tissue. A sequence of programmed cycles of waves may be applied to the subject (e.g., patient whole body, a body part, a portion of the patient body) at a frequency in a range of about 20-250 kHz, with low intensity for a long period of time (e.g., hours) so as to induce a reversal of aging in the target cells (e.g., senescent cells) while promoting or preserving growth of normal cells of the subject being treated. The programmed duty cycles of waves can alternate in the order of seconds, and the inventors' tested modulations between 0.1 Hertz and 10 Hz. The sound carrier frequency—as justified above—will fall in the range of 10 kHz to some 250 kHz for large tissue sections but can be several MHz for localized regions. The periodic forces imparted on the subject (e.g., internal organelles) mechanically distort the target/normal cells such that a mechanically-induced aging reversal process is triggered in the target cells. In some cases, the frequency and magnitude of the periodic forces, or the period of time of a treatment session may be determined based on the target disease and/or the body part/portion to be treated (e.g., whole body, arm, leg, breast, etc.).

As used herein, the term “spontaneous cell death” or “apoptosis” includes but is not limited to apoptosis, autophagy, and certain forms of necrosis. In the present disclosure, the spontaneous cell death may be caused by a periodic and repetitive sequence of forces which is different from cells death caused by increased amplitude or intensity as in the conventional ablation treatment. Specifically, as used herein, the term “apoptosis” refers to a regulated series of biochemical events that eventually lead to cell suicide, and is characterized by readily observable morphological and biochemical phenomena, such as fragmentation of the deoxyribonucleic acid (DNA), condensation of the chromatin, chromosome migration in cell nuclei, the formation of apoptotic bodies, mitochondrial swelling, and the like.

The terms “subject,” “individual,” “user” and “patient” are used interchangeably herein to refer to a vertebrate, preferably a mammal such as a human. Mammals include, but are not limited to, murines, simians, humans, farm animals, sport animals, and pets. Tissues, cells and their progeny of a biological entity obtained in vivo or cultured in vitro are also encompassed. In some cases, the subject may suffer from senescence or aging.

The target cells for the methods of the present disclosure can be any cells in need for treatment for aging, senescence, or wound healing. In some embodiments, the target cell is a senescent cell. The target cells may include senescent cells in different areas of the body of a patient. The senescent cells that can be used in the method of the present disclosure include but are not limited to prostate cells, breast cells, colon cells, lung cells, head & neck cells, brain cells, bladder cells, lymphocytes, ovarian cells, renal & testis cells, melanocyte cells, liver cells, cervical cells, pancreatic cells or gastrointestinal cells. In some cases, the target cells may be cells in a diseased tissue that desire regeneration, growth, repair, and the like.

In some embodiments, the mechanotherapy may induce or regulate apoptosis of the target cell by exposing the target cells to periodic stretch/pressure forces with pre-determined characteristics. In some embodiments, the method of the present disclosure at least partially stimulates, increases, opens, activates, facilitates, enhances activation, sensitizes, or upregulates apoptosis signaling pathway of tumor cells. The mechanotherapy may effectively activate the cell surface receptors involved in apoptosis signaling pathway of tumor cells. The cell surface receptors may be capable of sensing mechanical cues, radiation, or waves. Immersion mechanotherapy may apply cyclic and/or structured remote forces such as ultrasound waves to the target tissue which effectively alters the tumor cells at the same time that it affects senescent cells.

The immersion mechanotherapy of the present invention does not induce a spontaneous cell death in normal cells. As used herein, the term “normal cell” refers to the basic healthy cell with normal functions to maintain correct functioning of tissues, organs, and organ systems. Normal cells may undergo spontaneous cell death as part of normal development, to maintain tissue homeostasis, and in response to unrepairable damage. The one or more cycles of waves may be applied to the normal cells without inducing spontaneous cell death of the normal cells in the tissue of the subject.

The various characteristics of the cyclic force such as intensities, frequencies, amplitude and the like may refer to the intensity, frequency or amplitude levels at the effective tissue site or the force imparted on the target cells. As an example, the force may comprise low intensity cycles applied to the tissue site over hours. In some cases, the cyclic force applied directly to the tissue site or target cells may be delivered with long irradiation times (e.g., hours) at low-frequency (e.g., 5-30 kHz, 30-250 kHz, 150 kHz to 1 MHz, etc.) and at low intensity levels (e.g., 5, 10, 15, 20, 25, 40, 50, 60, 70, 75, 100, 125, 150, 175, 200, 250, 300, 350, 400, 450, <500 mW/cm² and ranges in between). The cyclic force, very generally, is mediated by the waves and depends of their waveform, or structured waveform and the like which are used interchangeably throughout the specification unless context suggests otherwise. The cyclic force may be transmitted to the subject while the subject is, e.g., immersed in a liquid contained in a tub, which may also include water that has been heated. As used herein, the “cyclic force” refers to force envelopes that are recurring or repetitive and manifest as a modulation of a basic pressure force envelope.

The waveform may be of arbitrary shape, as long as it achieves a uniform or reasonably uniform membrane stress or stress envelope on the target cell/normal cell. In many cases, the waveform may approximate a sine wave. When such ultrasound waves are applied to the target cell/normal cell, the cell shape changes from a lateral ellipsoid shape to a longitudinal shape and then springs back in a cyclic manner while the stress on the membrane may remain relatively constant if frequency and amplitude match.

In some cases, the waveforms may be determined based on the type of target cells. For instance, the waveforms delivered to the target tissue may be different according to the different types of cells or tissue to be treated. In some cases, the waveforms delivered to the target tissue may be dependent on the mechanical properties of the target cell (e.g., dynamic and kinematics properties, diameter, rigidity, or inertia of a cell, etc.).

The wave applied to the subject may, or may not be direction-sensitive. Ultrasound of an optimal frequency range with pre-determined amplitudes may expose target cells/normal cells to cyclic deformations due to acceleration and sound pressure being of comparable strength. For example, when the ultrasound is applied to the cells at a relatively low frequency (e.g., 20-250 kHz), the cell shape may change from a lateral ellipsoid shape to a longitudinal shape then spring back in a cyclic manner while the stress on the membrane may remain relatively constant. Such kind of rapid deformation may also be referred to as diffuse forces as they do not have specific direction other than being circumferential to the cell. This does beneficially allow for an immersed and uniform therapy that does not require accurate alignment or orientation of the ultrasound device with respect to the subject. For example, for any portion of the subject immersed in the liquid of the tub, the administered ultrasound waves may not induce side effects on the normal cells while the efficacy of the treatment may be substantially the same.

In some cases, the wave transmitted to and experienced at the site of the target tissue or target cells may be in a low intensity range because the energy absorbed by the subject will be only a fraction of the energy emitted by the transducer. For example, low intensity may be no more than 450 mW/cm², 400 mW/cm², 350 mW/cm², 300 mW/cm², 250 mW/cm², 200 mW/cm², 150 mW/cm², 100 mW/cm², 90 mW/cm², 80 mW/cm², 75 mW/cm², 70 mW/cm², 60 mW/cm², 50 mW/cm², 40, mW/cm², 30 mW/cm², 25 mW/cm², 20 mW/cm², 15 mW/cm², 10 mW/cm², or any number below 450 mW/cm² or above 10 mW/cm². With low intensity, the inventors refer to the general diagnostic average absorbed power limit of 100 mW/cm² which is considered harmless for nearly all tissues sans fetuses and corneas.

In some cases, the sound waves at the site of the target tissue or target cell may have frequencies in an optimal range based on the mechanical properties of the target cell. For example, the frequency may be in the range from about 5 kHz to 30 kHz, 30 kHz to about 100 kHz, 30 kHz to about 150 kHz, 30 kHz to about 200 kHz, 30 kHz to about 250 kHz, 40 kHz to about 100 kHz, 40 kHz to about 150 kHz, 40 kHz to about 200 kHz, 40 kHz to about 250 kHz, 50 kHz to about 100 kHz, 50 kHz to about 150 kHz, 50 kHz to about 200 kHz, 50 kHz to about 250 kHz, 60 kHz to about 100 kHz, 60 kHz to about 150 kHz, 60 kHz to about 200 kHz, 60 kHz to about 250 kHz, 70 kHz to about 100 kHz, 70 kHz to about 150 kHz, 70 kHz to about 200 kHz, 70 kHz to about 250 kHz, 30 kHz to about 210 kHz, 30 kHz to about 220 kHz, 30 kHz to about 230 kHz, 30 kHz to about 240 kHz, 40 kHz to about 210 kHz, 40 kHz to about 220 kHz, 40 kHz to about 230 kHz, 40 kHz to about 240 kHz, 50 kHz to about 210 kHz, 50 kHz to about 220 kHz, 50 kHz to about 230 kHz or 50 kHz to about 240 kHz, 150 kHz to 1 MHz, and levels of ultrasound frequency within these stated frequencies.

In some embodiments of the present disclosure, the energy may be delivered by modulating it. In some cases, the amplitude of the sound is sufficient to induce cell shape deformation by a certain amount without introducing mechanical damage to the target cells or normal cells. For example, the target cell shape may be deformed by from about 1% to about 5%, from about 3% to about 8%, from about 5% to about 10% when irradiated. For instance, a given time interval, the on-off ratio of the irradiation—called duty cycle—may be about 5%, 10%, 15%, 20%, 25%, 30%, 35%, 40%, 45%, 50%, 55%, 60%, 65%, 70%, 75%, 80%, 85%, 90%, 95%, and the like. The duty cycle is defined as the fraction of time that the signal is “on” (e.g., transmitted) per time unit. The intensity or the amplitude of the wave will be determined such that no thermal damage is introduced in the target tissue or to the subject.

The cyclic force may be applied to the target tissue or cells over a period of time. The period of time may be in the range of hours. For example, in an immersion therapy, waveforms may be delivered to the subject as a treatment session for about 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 15 hours, 20 hours, 24 hours, 48 hours or more. A treatment session may be an exposure to radiation that is continuous or intermittent. A treatment session may include one or more sub-sessions that may or may not utilized the same cyclic force or waveform. In some cases, the treatment may be repeated for the same or a different length of time, one or more times for days, weeks, months, years, or for the life of the subject. The treatment may be continuously conducted while the subject is sitting in the hot tub. Alternatively, or in addition to, the treatment may be repeated with pre-determined time intervals in between.

As used herein, the plurality of characteristics of the waves such as intensities, amplitude and frequencies are the intensity, amplitude and frequency levels at the effective tissue site, not the actual output value of the ultrasound transducer. In some cases, one or more characteristics of the wave as direct output from the ultrasound transducer may be different from those of the cyclic force effective at the target tissue or target region.

The output of the ultrasound transducer for generating such a waveform may have a greater intensity level than the resulting effective amount at the target tissue site to account for energy loss or dispersion during transmission. For example, a certain amount of energy is absorbed or scattered by the biologic tissue (e.g., skin, bone, muscle, and underlying fascia) and the liquid in the tub while the ultrasound traverses tissue and liquid before it reaches the target tissue/region. Owing to the low frequency characteristic, the intensity loss may be low and the penetration depth may be long. As an example, no more than 5%, 10%, 15%, 20%, 25%, 30%, 35%, 40%, 45%, 50%, 55%, 60%, 65% of energy may be absorbed when transmitting the waves. This advantageously allows for an immersion mechanotherapy as waves that can be effectively transmitted to an internal tissue of an individual through a pool/container of liquid and surrounding organs. Moreover, owing to the low frequency and low intensity characteristics of the waves, the heat generated as a result of the wave penetrating tissues was calculated to be insignificant based upon experimental values of heating versus power at these wavelengths.

The waves may be impinged on the subject in the form of an immersion mechanotherapy. In some embodiments, at least a portion of the body of the subject is immersed in a liquid contained in a tub/vessel. Such portion of the body of the subject can be, for example, 0.5/6-100% of body of the subject. In particular, the subject can have 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95% or 100% part of the body immersed in the liquid. The disclosed ultrasound waves may penetrate through some or all of the parts of the body immersed in the liquid without inducing side effects or damage to the normal cells/tissues.

Mechanotherapy Systems and devices. In one aspect, the present disclosure provides devices and systems configured for implementing the mechanotherapy described above in a subject in need thereof. In some cases, the devices or systems may be handheld, held and/or controlled by a robotic arm, comprise a vessel or container for containing a liquid in which at least a portion of an individual/subject is immersed, and one or more ultrasound transducers capable of producing one or more programmed cycles of waves for a determined period of time to treat the senescent cells. The vessel or container may be sized and shaped to hold one or more individuals. In some cases, the vessel or container may comprise an interior shape or geometries to facilitate transmitting the waves to the individual.

The present invention can use one or more ultrasound transducers capable of producing one or more programmed outputs for a pre-determined period of time to treat an individual. The disease may be a wound of the individual. The one or more transducers may be configured to generate structured waves as described elsewhere herein. The structured waves can be focused or unfocused. The ultrasound transducer(s) may be a single element or an array of ultrasound transducers, or a complex assembly of emitters.

In one example, the one or more transducers may be an array of ultrasound transducers. The array of ultrasound transducers may be directed at one or more tissues in the body. In some cases, the plurality of ultrasound transducers may be mounted at spaced locations on the side wall and/or the bottom of a container or vessel. In some embodiments, the one or more transducers may be displaced in the inside space of the vessel using a waveguide. In some embodiments, the one or more transducers may be displaced outside the vessel. In some embodiments, the one or more transducers may be positioned close to the bottom wall of the vessel. In some embodiments, the one or more transducers may be positioned close to the side wall of a vessel. In some embodiments, the one or more transducers may be immersed in a liquid in a vessel. The one or more transducers may be arranged into desired locations (e.g., with optimal spacing, orientation with respect to each other) such that when the transducers operate concurrently, ultrasound waves may be generated collectively to achieve a desired effect (e.g., direction, focal plane, intensity, etc.).

In some embodiments, the one or more transducers may be packaged and sealed in a panel for sterilization purpose. The panel may have a substantially smooth surface and may be composed of a material that can be sterilized by normal methods that are compatible with the tub, such as steam, heat and pressure, chemicals, UV light and the like. In some embodiments, the panel surface may be disposable.

The panel may be removably coupled to the one or more transducers. In some cases, the panel may be composed of materials and may comprise geometrics (e.g., thin-walled or sheet) so as to reduce the wave resistance. In some cases, the panel may comprise docking features or structures to mate with a shape of the one or more transducers thereby providing a snug fit.

The one or more ultrasound transducers may or may not be in direct contact with the liquid. The one or more ultrasound transducers may or may not be in direct contact with the bathtub. When an individual is positioned, the individual may or may not be in direct contact with the one or more ultrasound transducers.

The one or more ultrasound transducers may be removably coupled to a device that is used to hold a subject, such as a chair, bed, or tub and/or may be permanently affixed to the device. In some cases, the one or more ultrasound transducers may be attached to a wall. For instance, a transducers array (such as a phased array) may be coupled to the wall of a bathtub and the direction of sound waves may be controlled using beamforming techniques.

The one or more ultrasound transducers may be sealed and water-proof. The one or more ultrasound transducers or the ultrasound device may be provided with an internal cooling system to stabilize an operation temperature of the one or more transducers. Any suitable cooling methods can be utilized for cooling the ultrasound device. The cooling method can be passive cooling such as by arranging the ultrasound probe to be thermally coupled to a heat sink or other cooling feature (e.g., heat pipe, heat spreader, etc.). Passive cooling may refer to dissipation of heat from an ultrasound transducer (e.g., ultrasound probe) by thermal contact with a heat sink or cooling fins. In some cases, coolant such as fluid coolant or gas coolant may be circulated over the surface of the ultrasound transducers, cooling fins and/or heat sinks to aid in passive cooling. The cooling method can be active cooling such as utilizing a thermoelectric cooler driven by temperature controller to adjust or stabilize the ultrasound transducers operating temperature.

In some embodiments, the one or more transducers may be carried by a robotic arm. The robotic arm may be configured to provide one or more degrees of freedom of motion to the one or more transducers. The one or more transducers may be controlled to be positioned at a desired location or orientation such as different portions of body may be treated. The location or orientation of the one or more transducers may be fixed during a treatment session. Alternatively, or in addition to, the one or more transducers may be controlled to move (e.g., sweep motion, positioned to different locations in different sub-sessions) during a treatment session.

In some cases, the robotic arm may be a gantry. The robot arm may be a 6-axis robot arm. The robot arm may be capable of motion about 1 or more, two or more, three or more, four or more, five or more, or six or more axes of motion. The robot arm may comprise one or more, two or more, three or more, four or more, five or more, six or more, seven or more, eight or more, nine or more, or ten or more joints. The joints may comprise motors that may allow various support members to move relative to one another. The robot arm may comprise one or more, two or more, three or more, four or more, five or more, six or more, seven or more, eight or more, nine or more, or ten or more support members. In one example, a first support member may bear weight of an end effector. A second support member may bear weight of the first support member and/or the end effector, and so forth. The motors may allow rotation of one or more support members relative to one another. One or more sliding mechanism may be provided that may allow lateral displacement. The robot arm may have a free range of motion that may match or exceed the range of motion of a human arm. Ball and socket joints may or may not be employed by the robot arm.

In some embodiments, the one or more transducers may be affixed to the end effector of the robotic arm. Position and orientation of the one or more transducers may be controlled by controlling the robotic arm. In some cases, the robotic arm may be controlled by a robotic controller. The robotic controller may be under the control of a controller that positions the one or more transducers. Regarding the control system, a cascaded proportional-integral-derivative (PID) may be used to control the attitude and velocity of the robotic arm. It should be noted that there are a variety of control algorithms that can be used to control a gimbal or carrier system, including but not limited to: ON-OFF, PID modes, feedforward, adaptive, intelligent (Fuzzy logic, Neural network, Expert Systems and Genetic) control algorithms. For a specific control model, such as PID control, the control system can be different according to different control objective/output variable (e.g., angular velocity, angular position, angular acceleration, or torque) and different input variable (e.g., input voltage). Accordingly, control parameters may be represented in various ways.

In some cases, the robotic arm controller may be configured to control the robotic arm using sensor data as feedback information. The sensor data may be related to the location of the end effector (i.e., the one or more transducers) with respect to a subject. In some cases, sensors such as proximity sensor or imaging sensor may be used to provide such location/proximity information. In some cases, the one or more ultrasonic transducers along with receivers may be provided as ultrasonic sensor to determine the proximity. In some cases, additional sensors may be included to provide such information.

In some cases, the robotic arm may automatically position the one or more transducers to an initial position. In some embodiments, the robot arm can be passively moved by a user. In such case, a user may push the arm in any position and the arm compliantly moves. The robotic arm can also be controlled in a compliant mode to improve human robot interaction. For example, the compliant motion control of the robot art may employ a collision avoidance strategy and the position-force control may be designed to save unnecessary energy consumption while reducing impact of possible collisions. The plurality of ultrasound transducers may operate collectively to generate a sequence of force pulses. The number of transducers may be any number such as a number from 1 to 1000, and may emit at different amplitudes and/or phase relationships. For instance, adjacent transducers may have a constant progressive phase shift or variable phase shift thereby adjusting the beam/wave direction. In some cases, the frequencies of the transducers may be all the same, or may emit different frequency ranges thereby providing a composite waveform including multiple frequency components.

The ultrasound may be focused, unfocussed or a combination of both. In some cases, additional elements such as acoustic lenses or reflecting mirrors may be utilized to produce focused ultrasound. The focal plane or focal length of the transducer (array) may be adjusted to direct the beam to the location of a target region in the subject. In some examples, the ultrasound device may comprise an array of individually controlled transducers that allows for beam steering and focusing. Beamforming techniques such as phased array beamforming or beam control methods such as using mirrors of moving acoustic lenses for adjusting focal length of the device may be utilized. The array of transducers may operate collectively to generate a waveform and transmit the waveform to a target location/region. In addition to the focal length of the ultrasound device, one or more characteristics of the wave such as frequency, duty factor, amplitude, intensity and the like may be modulated by controlling the array of ultrasound transducers.

Alternatively, or in addition to, the ultrasound may be unfocussed. Unfocussed ultrasound wave may travel through a liquid and/or biological tissues immersed therein. The unfocussed ultrasound may be applied to a diffuse large area/portion of the subject. In some embodiments, the one or more transducers may be configurable such that the provided ultrasound system may be capable to switch between focused ultrasound mode and unfocussed mode, or to operate in dual mode.

In some cases, the one or more transducers may be customized to be a neutral generator to provide additional user safety. For example, with use of transducer or antenna arrays whose feed net voltages add up to zero or near zero, safe human contact may be provided even in the case of a failure of the insulation, as liquid ingress will have to cross a zero volt contact before reaching the internal part of a transducer. The array of antenna elements can be built with alternating coil direction or transducers with inverted piezo crystals thereby allowing for a zero net voltage. Such alternating direction or inverted piezo crystals design can be applied to one or more pairs of the antenna elements or the entire antenna array.

In some embodiments, the tub liquid may be controlled to be at temperature between 4, to 45, between 16 to 20, between 20 to 40, between 36 to 40 degrees Celsius or any other temperature range based on a user preference. In some embodiments of the present disclosure, the immersion mechanotherapy is delivered in the form of tub or spa and a heater may be involved to elevate the water temperature to a level typically in the range of about 95-105 degrees F. The transducer system may be part of the heating. In some embodiments, the system may comprise a temperature controlling system. The temperature controlling system may comprise one or more temperature sensors and/or temperature controller for controlling the temperature of the medium (e.g., a liquid) as needed. The one or more temperature sensors can be disposed at any suitable location with respect to the bathtub. The temperature can be manually adjusted by an individual, a user, an operator or be automatically controlled according to a pre-programmed therapy plan.

In some embodiments of the present disclosure, the system may also comprise features for cooling an overheated medium or bathtub liquid. For example, water pumping, or recycling may be used to keep the water temperature stable. Such features can be the same as those known in typical spa or massage devices. For instance, the water may be filtered and heated, and may be normally recycled to the spa through one or more hydrotherapy massage jet nozzles mounted at spaced locations on the side wall. Care will have to be taken to avoid air bubbles, since they will absorb the ultrasound and make the system ineffective.

The medium used in the mechanotherapy of the present disclosure can be any medium that can transduce the ultrasound of the present disclosure. In some embodiments, the medium is a liquid. In some embodiments, the medium is a gel. In some embodiments, the medium is water. In some embodiments, the medium is alcohol or saline water.

In some embodiments, the liquid may be water. The liquid can be any suitable liquid that is safe to human contact and has an acoustic impedance similar to human tissue. For example, the acoustic impedance may be similar to, or higher than, that of the tissues/skin of the subject to be treated. In some cases, chemicals may be added to the water. The chemical that can be used in the medium can be any chemicals that do not prohibit the transduction of the wave of the present disclosure. In some cases, the chemical is a salt. In some cases, chemical agents, such as chlorine may be periodically added to the spa water in prescribed amounts suitable for preventing growth of bacterial organisms, to maintain the water in a hygienic state. Other chemical agents such as a sanitizer, e.g., an oxidizer such as chlorine, may also be periodically added to the water.

In some embodiments, a part of the body of the subject is immersed in the tub liquid. The part of the body of the subject can be, for example, 0.5%-100% of the body of the subject. In particular, the subject can have 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95% or 100% part of the body immersed in the liquid.

In some embodiments, a vessel or tub may comprise an open-top-end enclosure including a bottom wall and one or more side walls. In some embodiments, the vessel may comprise a bottom wall, opposed sidewalls and opposed end walls. In some embodiments, the vessel may further comprise an openable top lid. The bottom wall and/or side wall of the vessel can be any appropriate shape. For example, the shapes of the bottom wall or side wall of the vessel may include but are not limited to round, oval, rectangular, square, trapezoidal, triangular or irregular shape. In some embodiments, an interior wall of the vessel may be shaped or have a pre-determined geometric configuration to reflect the ultrasound wave into the immersed part of the subject or a target location. In some cases, an interior wall of the vessel may be shaped or composed of US-absorbing materials to regulate the wave field such that undesired waves (e.g., waves reflected off the interior wall) may be reduced. This beneficially allows for controlled waves of a desired frequency and intensity to be received and effective at the immersed body. In some embodiments, the vessel can be selected from a tub, a bucket, a tank, a container, and a pool. In some embodiments, the vessel can be a tub. In some embodiments, the vessel is a spa tub. In some embodiments, the vessel is a swimming pool. The material of the tank wall can be any appropriate type of material including but not limited to glass, metal or ceramic, aluminum and steel, fiber glass, plywood, porcelain and the like. In some embodiments, material of the immersion element may be a high temperature ceramic.

In some embodiments, the system may further comprise a user positioning system. In some cases, the user positioning system may utilize proximity sensors to detect the location of the body of the user. The proximity sensors may be the ultrasound device where the ultrasound transducer may be paired with one or more receivers to measure a distance based on time of flight. Alternatively, or in addition to, additional sensors may be used to locate the body of the subject. For instance, additional proximity sensors (e.g., ultrasonic sensors, cameras) may be used to detect the position of the subject with respect to the vessel/transducer or the proximity of the subject to the transducers.

The controller may control the one or more ultrasound transducers coupled to the vessel. The system may further comprise a computer system and one or more databases operably coupled to the controller over a network. The computer system may comprise a therapy planning module implementing methods provided herein for generating therapy plans.

The computer system may be used for generating a personalized therapy plan based on personal/user information, device setup, diagnostic information, and the like. Although the illustrated diagram shows the controller and computer system as separate components, the controller and computer system can be integrated into a single component.

For instance, a therapy plan may comprise information about level of mechanotherapy (e.g., frequency, intensity, amplitude, duty cycle of the ultrasound waves), type of therapy (e.g., a reduction in senescent cells, reduction in cellular senescence, or wound healing), information about the treatment region (e.g., location, volume, tissue type, etc.), operation settings (e.g., temperature control, focused/unfocused beam), treatment duration, user information (e.g., user preferred spa temperature) or others.

A therapy plan may be generated in a fully automated, semi-automated, or manual fashion. In some cases, the therapy plan may be generated automatically upon receiving a diagnostic input or user information. For instance, the frequency, amplitude, intensity of the ultrasound waves to be delivered may be determined automatically based on the diagnostic information (e.g., tissue location, volume, disease type, application purpose) and/or user information. In some cases, the treatment plan may be generated using AI techniques and or machine learning methods. For instance, machine learning models may be trained for generating a therapy plan. In some cases, the input data supplied to the machine learning model may include diagnostic information, device information, personal information or others as described elsewhere herein. In some cases, the output of the machine learning model can be a therapy plan or one or more parameters of the treatment (e.g., characteristic of forces, device setup, spa duration, etc.). The therapy plan may dynamically adapt to real-time condition based on feedback information. Alternatively, or in addition to, the therapy plan may run through the entire course without real-time feedback information.

In some cases, the machine learning method used for generating the treatment plan may comprise one or more machine learning algorithms. Examples of machine learning algorithms may include a support vector machine (SVM), a naive Bayes classification, a random forest, a deep learning model feedforward neural network, radial basis function network, recurrent neural network, convolutional neural network, deep residual learning network, or other supervised or unsupervised learning algorithm.

The controller may be operated to provide the ultrasound device controller information about a pulse sequence and/or to manage the operations of the entire system, according to installed software programs. In some cases, the controller may also serve as an element for instructing a subject/user to perform tasks, such as, for example, positioning a part of the body to a given location in a vessel by a voice message produced using an automatic voice synthesis technique. The controller may receive commands from an operator which indicate the mechanotherapy to be performed. Alternatively, the system may be for home-use and the user may receive instruction via a user interface (e.g., mobile application) operably coupled to the computer system via a network. The controller may comprise various components such as a pulse generator module which is configured to operate the system components to carry out the desired wave or cyclic force sequence, producing data that indicate the timing, strength and shape of the wave or ultrasound pulses to be produced, and the direction of the beam. In some cases, the controller may control pulse generator module and/or a set of gradient amplifiers of the transducers to control the frequency, amplitude and shape of the pulses or waves to be produced during the therapy. In some cases, the controller may control the phase shifting of phased array transducers to adjust the beam direction, focus, and other properties of the ultrasound waves. In some cases, the controller may control a robotic arm carrying the one or more transducers thereby controlling the orientation and location of the one or more transducers with respect to the subject.

In some situations, the controller may also receive real-time patient data from a physiological acquisition controller that receives signals from sensors attached to the patient, such as ECG (electrocardiogram) signals from electrodes or respiratory signals from bellows. The controller may be coupled to various sensors for monitoring the condition of the patient (e.g., wound healing progress), the ultrasound transducers, and the vessel (e.g., liquid filling, liquid temperature, etc.). For instance, a temperature sensor may be coupled to the controller for temperature control during the operation. In some cases, the system may include a user positioning system that may receive commands to instruct the user to move to a desired location for the treatment or to immerse a certain body part in the bathtub water.

In some cases, the controller may comprise or be coupled to an operator console (not shown) which can include input devices (e.g., keyboard) and control panel and a display. In the home-use situations, the operator console may be a user interface. For example, the controller may have input/output (I/O) ports connected to an I/O device such as a display, keyboard and printer. In some cases, the operator console may communicate through the network with the computer system that enables an operator to control the therapeutic procedure or modify a therapeutic plan on a screen of display. In some cases, a user may be allowed to view the disease progress such as wound healing progress on the display.

The system may comprise a user interface. The user interface may be configured to receive user input and output information to a user. The user input may be related to the control of a therapeutic procedure (e.g., wound healing, tissue repair, tissue regeneration, etc.), generating/modifying a therapeutic plan (e.g., select body part to be treated), control of the spa settings (e.g., temperature, massage modes, etc.), and the like. The user input may be related to the operation of the vessel (e.g., massage modes such as therapy level, water temperature, etc.), operation of the ultrasound waves (e.g., parameters for controlling the waves to be delivered to the target region such as frequencies, amplitude, duration, etc.). The user input may be related to various operations or settings for generating a therapeutic plan. The user interface may be rendered on a screen such as a touch screen or any other user interactive external device such as handheld controller, mouse, joystick, keyboard, trackball, touchpad, button, verbal commands, gesture-recognition, attitude sensor, thermal sensor, touch-capacitive sensors, foot switch, or any other device, or be implemented via an app or a software package.

The system may comprise computer systems and database systems, which may interact with the controller or form the controller. The computer system can comprise a laptop computer, a desktop computer, a central server, distributed computing system, etc. The processor may be a hardware processor such as a central processing unit (CPU), a graphic processing unit (GPU), a general-purpose processing unit, which can be a single core or multi core processor, a plurality of processors for parallel processing, in the form of fine-grained spatial architectures such as a field programmable gate array (FPGA), an application-specific integrated circuit (ASIC), digital signal processor (DSP), and/or one or more embedded processors. The processor can be any suitable integrated circuits, such as computing platforms or microprocessors, logic devices and the like. Although the disclosure is described with reference to a processor, other types of integrated circuits and logic devices are also applicable and especially dedicated processors for different functions. The processors or machines may not be limited by the data operation capabilities. The processors or machines may perform 512 bit, 256 bit, 128 bit, 64 bit, 32 bit, or 16 bit data operations.

The system may comprise one or more databases. The one or more databases may utilize any suitable database techniques. For instance, structured query language (SQL) or “NoSQL” database may be utilized for storing diagnostic data, such as image data obtained by suitable imaging modalities, training datasets or trained model for generating therapeutic plan, parameters of a therapeutic plan, historical therapeutic plan, user-preferred spa setting, etc. Some of the databases may be implemented using various standard data-structures, such as an array, hash, (linked) list, struct, structured text file (e.g., XML), table, JSON, NOSQL and/or the like. Such data-structures may be stored in memory and/or in (structured) files.

In another alternative, an object-oriented database may be used. Object databases can include a number of object collections that are grouped and/or linked together by common attributes; they may be related to other object collections by some common attributes. Object-oriented databases perform similarly to relational databases with the exception that objects are not just pieces of data but may have other types of functionality encapsulated within a given object. If the database of the present disclosure is implemented as a dedicated system, the use of the database of the present disclosure may be integrated into another component such as the component of the present invention. Also, the database may map a mix of data structures, such as objects and relational structures. Databases may be consolidated and/or distributed in implementations. Portions of the data in the database, e.g., tables, may be exported and/or imported and thus decentralized and/or integrated/migrated.

The network may establish connections among the components in the system and a connection of the system to external systems. The network may comprise any combination of local area and/or wide area networks using both wireless and/or wired communication systems. For example, the network may include the Internet, as well as mobile telephone networks. In one embodiment, the network uses standard communications technologies and/or protocols. Hence, the network may include links using technologies such as Ethernet, 802.11, worldwide interoperability for microwave access (WiMAX), 2G/3G/4G mobile communications protocols, asynchronous transfer mode (ATM), InfiniBand, PCI Express Advanced Switching, etc. Other networking protocols used on the network can include multiprotocol label switching (MPLS), the transmission control protocol/Internet protocol (TCP/IP), the User Datagram Protocol (UDP), the hypertext transport protocol (HTTP), the simple mail transfer protocol (SMTP), the file transfer protocol (FTP), and the like. The data exchanged over the network can be represented using technologies and/or formats including image data in binary form (e.g., Portable Networks Graphics (PNG)), the hypertext markup language (HTML), the extensible markup language (XML), etc. In addition, all or some of links can be encrypted using conventional encryption technologies such as secure sockets layers (SSL), transport layer security (TLS), Internet Protocol security (IPsec), etc. In another embodiment, the entities on the network can use custom and/or dedicated data communications technologies instead of, or in addition to, the ones described above.

### Example 1. Senescence Induction and Characterization

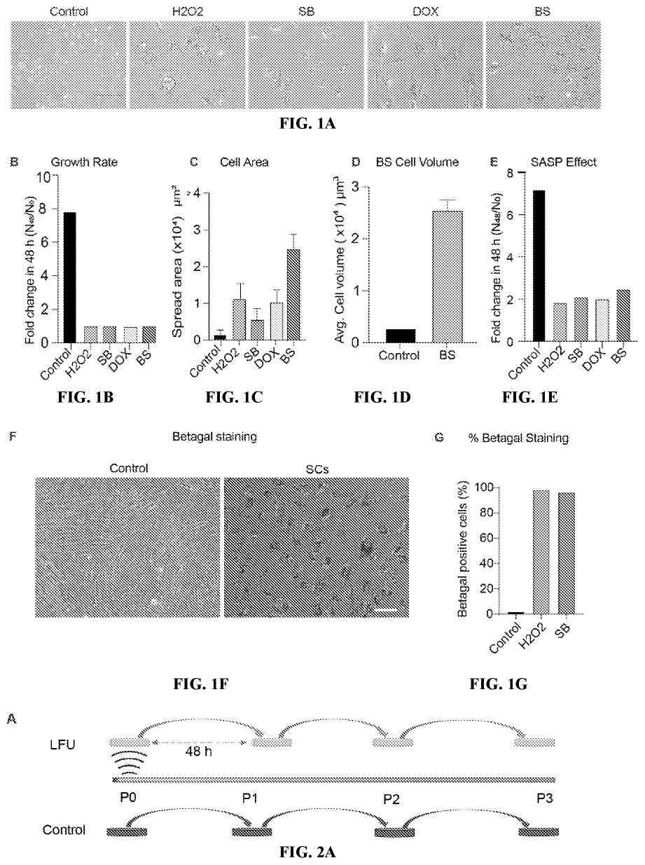

To test the generality of any senescent treatment, the inventors used four different methods to cause Vero cell senescence, replicative, hydrogen peroxide, sodium butyrate and Bleomycin sulfate. Senescence was measured as a decreased growth rate, increased O-gal activity, increased cell size, and secretion of pro-inflammatory factors called SASP. All four criteria were met with each type of senescent cells (FIGS. 1A to 1G). In each of those cases, cell growth was effectively blocked, and residual growth was minimal. (FIG. 1B). There were significant differences in the size of the senescent cells with bleomycin sulfate-treated cells being the largest and replicative senescent cells the smallest (FIGS. 1C and 1D). To assay for SASP activity, the inventors cultured the senescent cells overnight and collected the supernatant from each dish. Supernatants were diluted 1:1 with fresh media and added to normal control cells. The change in normal cell number was measured after a 48 hour incubation. The supernatant from the senescent cells inhibited growth of the normal cells as expected for SASP (FIG. 1E). When β-galactosidase levels were measured by antibody staining, there was a relatively uniform increase in levels (FIGS. 1F and 1G). Thus, by all four criteria, the four different types of senescent cells were indeed senescent.

FIGS. 1A to 1G show the characterization of senescent cells. (FIG. 1A) Brightfield images of senescent cells induced by H₂O₂, sodium butyrate (SB), Doxorubicin (Dox), and Bleomycin Sulphate (BS). Scale bar=300 μm. (FIG. 1B) Quantification of proliferation shows no senescent growth after a 48 hours incubation. (FIG. 1C) Senescent cells become enlarged compared to the normal control cells. (FIG. 1D) Quantification of avg. cell volume of bleomycin sulfate treated cells compared to control cells. (FIG. 1E) Conditioned medium from senescent cells inhibits the growth of normal proliferating cells. (FIG. 1F) SA-β-galactosidase staining of control (proliferating) and BS treated senescent cells. Scale bar=300 μm. (FIG. 1G) Level of β-galactosidase senescence marker in H₂O₂ and SB induced SCs. Scale bar=300 μm. Data in (FIG. 1B-1H) Minimum of 150 cells were analyzed for spread area and cell volume from n=2 experiments.

### Example 2. Low Frequency Ultrasound (LFU) Irradiation Activates Growth and Reversal of Senescent Phenotypes

To determine if mechanical activity could reverse some of the phenotypes of the senescent cells, the inventors irradiated senescent cells with low frequency ultrasound at low to intermediate power levels and modulation frequencies of about one Hz and a duty cycle of 50%. After irradiation for twenty minutes at 30 kHz, the cells were cultured for 48 hours.

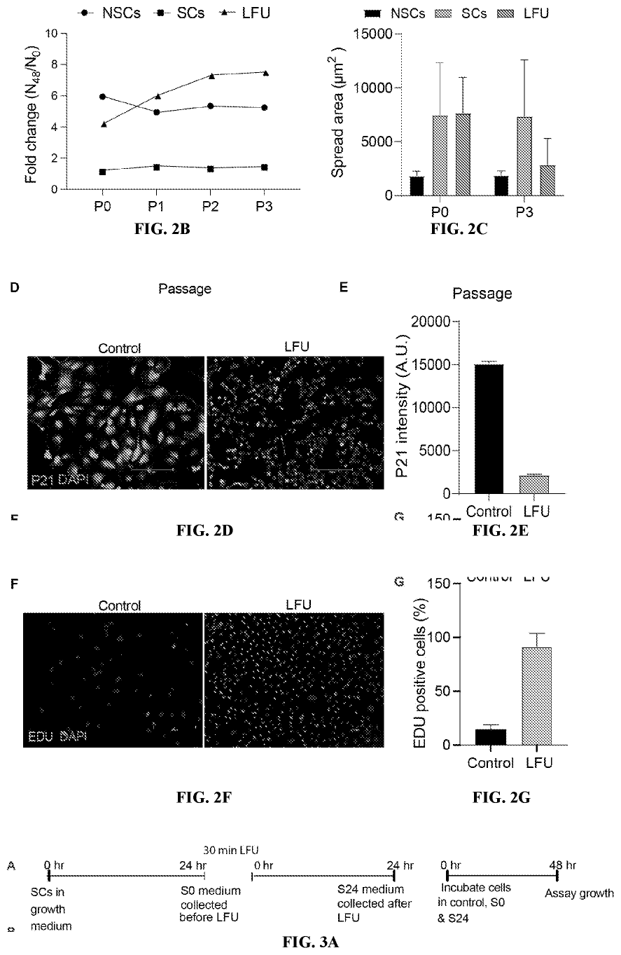

FIGS. 2A to 2G show that LFU reverses cell senescence. (FIG. 2A) Schematic illustration of senescence reversal experiment. SB treated Vero cells were treated with LFU and passaged after every 48 h for 8-10 days. (FIG. 2B) Graph showing growth of cells as fold change over 48 hours. Treated cells were passaged from P0 to P3 every 48 hours. (FIG. 2C) Cell area of LFU treated senescent cells is largely restored to normal by P3. (FIG. 2D) Representative anti-p21 immunofluorescence images of P3 control and LFU treated cells. Scale bar=300 μm. (FIG. 2E) Quantification of fluorescence intensity of p21 stained control and LFU treated P3 cells. Shown as mean±SD for >200 cells in each condition. (FIG. 2F) LFU increased proliferation as determined by EDU staining. (FIG. 2G) Quantification of EDU positive P3 cells. All graphs were plotted by mean±SD. Minimally 200 cells were analyzed in each condition.

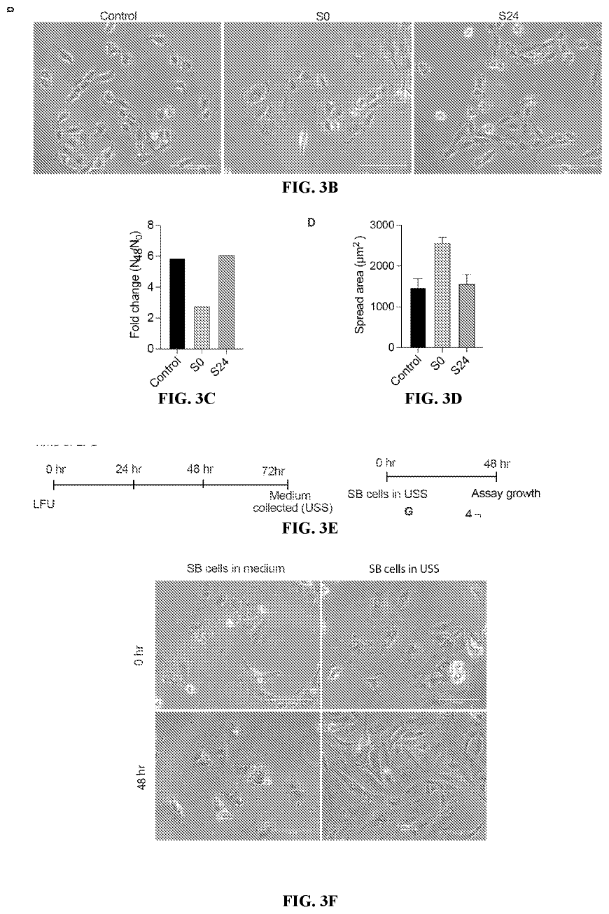

In the case of cell growth, the cells continued to grow for many days. The rate of growth did not diminish and thus, the cells appeared to have been restored to a normal state. In the case of the increase in cell size, ultrasound treatment caused an increase in the fraction of small cells that significantly overlapped with the size of the normal cells. After growth for 12 days, the fraction of small cells increased, indicating that the larger cells had a slower growth rate. In the case of the secretion of SASP, the cells initially produced a supernatant that dramatically inhibited growth of control cells and caused a significant increase in cell size; therefore, we tested if the secretion of SASP ceased after LFU (diagrammed in FIG. 3A).

The supernatant collected after LFU treatment had no effect on growth or spread area compared to the supernatant collected from the same cells before LFU treatment (FIGS. 3B-3D). In addition, the inventors also checked for cell scratch wound healing in the presence of the supernatant from senescent and treated senescent cells. Whereas the senescent cell supernatant inhibited wound healing, the supernatant from ultrasound treated cells did not. Thus, the reversal of the senescent cell state by ultrasound can be partial; however, the rejuvenated cells will dominate over time to essentially give a restored population of normal cells.

### Example 3. LFU Irradiation Activates Normal Cells to Secrete of Growth-Promoting Factors

Many studies have shown that physical exercise delays tissue and brain aging. To test the possibility that LFU could benefit normal cells and their senescent neighbors, the inventors treated normal cells with LFU over a period of three days (1 hour per day) and then collected the supernatant (FIG. 3E). The supernatant after this period was added to senescent Vero cells. Surprisingly, the senescent cells were rejuvenated by a 1:1 mixture of USS (ultrasound normal cell secretions) medium and normal medium. USS activated growth (FIG. 3F) and caused a decrease in the spread area of senescent cells (FIGS. 3F & 3G). This showed that ultrasound treatment might stimulate secretion of growth molecules from normal cells that would further benefit tissues where there was a mix of normal and senescent cells. Thus, there is every indication that ultrasound treatment will benefit tissues in aging organisms where there is the expectation that a mixture of senescent and normal cells will be present.

FIGS. 3A to 3H show that senescence associated secretory phonotype (SASP) inhibits control, but USS increases senescent cell growth. (FIG. 3A) Schematic illustration of the experiment to show that LFU blocks SASP secretion. SCs were cultured in growth medium for 24 hours then they were treated with LFU for 30 minutes. Supernatant was collected after the LFU treatment (S0) and again supernatant was collected after another 24 hours of incubation (S24). To check the effect of LFU treatment, supernatants S0 and S24 were used to check the growth of non-senescent control cells. (FIG. 3B) Representative brightfield images of control cells after 48 hours of incubation in normal growth medium, S0, and S24. Quantification of cell numbers (FIG. 3C) and cell areas (FIG. 3D) after 48 in control, S0 or S24 supernatants. (FIG. 3E) Timeline and strategy of LFU treatment of normal proliferating cells. Schematic showing that control cells were treated with US four times in the same media and the supernatant was collected (USS) for incubation of senescent cells for 48 hours. (FIG. 3F) Brightfield images show changes in morphology of senescent cells (SCs) in supernatant collected from LFU treated control cells and after 48 hours in response to USS. Senescent cells in normal growth medium were the controls. (FIG. 3G) Graph shows SCs in USS had increased growth and (FIG. 3H) decreased spread area. Graphs were plotted by mean±SD. Minimum 200 cells were analyzed for graph (FIG. 3C) and (FIG. 3H). Scale bar=300 μm.

### Example 4. LFU Influences Mitochondrial Dynamics and Lysosome Levels

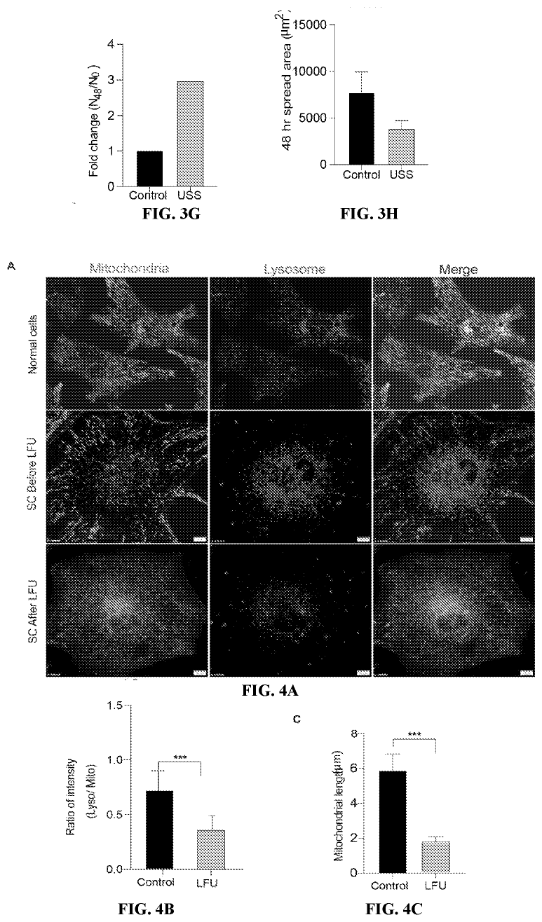

Since senescence is associated with an increase in mitochondrial fusion, the inventors followed the changes in mitochondrial length with senescence and the reversal of senescence with ultrasound. For comparison, the inventors also checked lysosome and microtubule morphologies since they have also been known to change in the senescence state. There was a significant increase in mitochondrial length and in the number of lysosomes in the senescent cells. Thus, we favor the model outlined in FIG. 4D, proposing that ultrasound acts by increasing autophagy through the inhibition of mTORC1.

FIGS. 4A to 4D show that LFU decreases mitochondrial length and lysosome intensity in senescent cells. (FIG. 4A) Representative immunofluorescent images of mitochondrial morphology and Lysosome fluorescence in normal, senescent and LFU-treated senescent cells stained with Mitotracker and lysosome tracker. Scale bar=10 μm. (FIG. 4B) Ratio of intensities of lysosomal to mitochondrial staining is decreased by LFU treatment of senescent cells. (FIG. 4C) Quantification of mitochondrial length shows decreased length after LFU. Results are shown as mean±SD, minimum 8 cells were analyzed, n>3 experiments and significance was determined using two-tailed unpaired t-test. ***p value<0.001. (FIG. 4D) Diagram of a working model for the rejuvenation of senescent cells by activation of autophagy through LFU inhibition of mTORC1 activity.

### Example 5. Reversal of Replicative Senescence

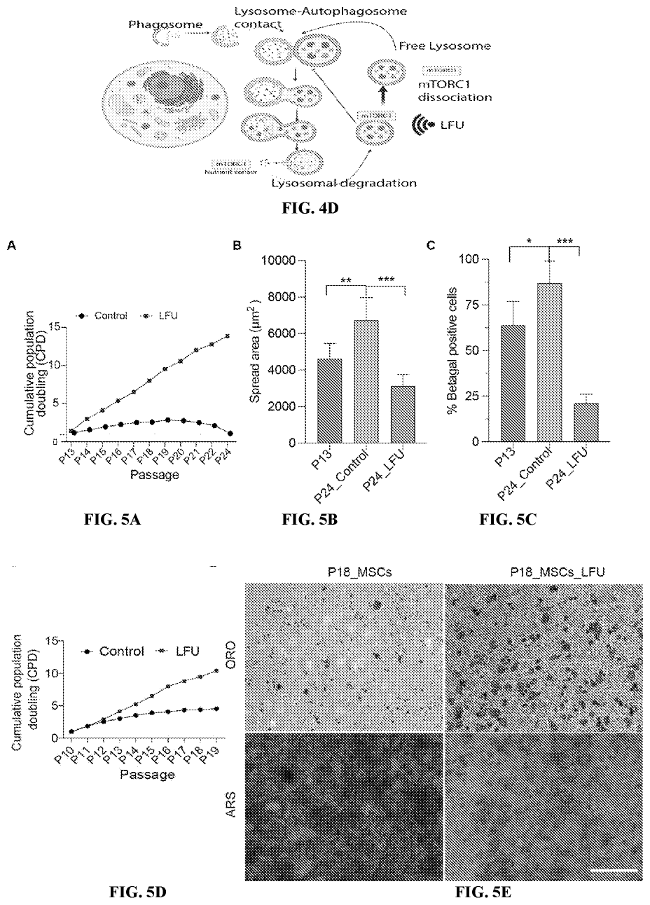

Because replicative senescence presumably reflects an inherent property of normal cells that limits growth, the inventors tested if ultrasound could effectively create an immortal population of cells. Human foreskin fibroblasts after P13 passages showed a significant increase in average cell size, a decrease in growth rate and an increase in β-galactosidase activity (FIG. 5A-C). However, after LFU treatment, the senescent cells grew like normal cells (FIG. 5A). Cells were treated with LFU after each passage and those cells continued to grow for more than P24 passages without a change in growth rate (FIG. 5A). Since the average time per cell division was 24-36 hours, this meant that the HFFs were able to grow more cells with LFU rejuvenation. After passage 24, LFU-treated cells were cultured on soft matrices, and they ceased to grow, indicating that the cells still exhibited rigidity-dependent growth. Thus, ultrasound reversal of senescence can effectively increase the replicative lifespan of cells without causing transformation or other apparent changes in cell phenotype.

To test if mesenchymal stem cells could also be expanded without altering their phenotypic behavior, cultures of mesenchymal stem cells at P10 were treated with LFU before each passage until P19. They showed significant growth expansion by 64-fold over control cells (FIG. 5D). However, when they were incubated in adipocyte differentiation medium, they adopted an adipocyte phenotype as indicated by oil red O staining. Alternatively, if they were incubated in osteocyte medium, they became osteocytes as indicated by Alizarin red S staining for calcium deposition. In both cases the LFU treated cells showed much more differentiation than the untreated controls (FIG. 5E).

FIGS. 5A to SE show the LFU reversal of replicative senescence increases number of cells. (FIG. 5A) Growth rate calculated as cumulative population doubling (CPD) for Control HFF and LFU treated HFF cells passaged every 48 hours from P13 to P24 passage and treated every other passage. (FIG. 5B). LFU treated cells were smaller than the p24 control and even p13 cells. (FIG. 5C) Number of SA-β-galactosidase positive cells decreased after LFU treatment. (FIG. 5D) Similarly LFU treatment of MSCs expanded the cell number P10-P19, with treatment every other passage. (FIG. 5E) LFU treated MSCs showed normal differentiation to (ORO) adipocytes and (ARS) osteocytes. Alizarin red S stained dye marked osteogenesis (ARS) and oil red o staining dye marked lipid droplets (ORO). Results are shown as mean±SD, minimally 200 cells for spread area and 150 cells for percentage β-galactosidase analysis, n>3 experiments, and significance was determined using two tailed unpaired t-test. ***p value<0.001, **p value<0.01, and *p value<0.05.

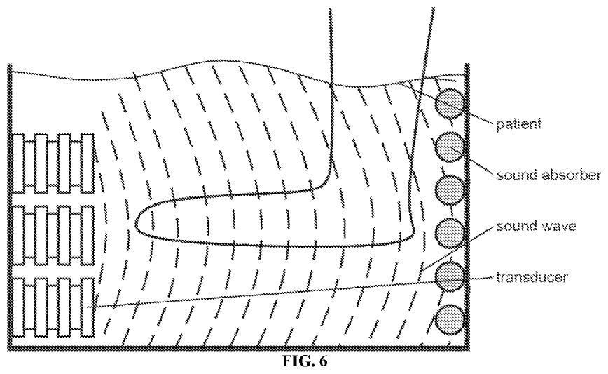

FIG. 6 is a side view of an US device showing the treatment of a foot using one type of the present invention. Transducers are depicted that send sound waves to the foot. The sound waves can then contact the sound absorbers. The skilled artisan will understand that any limb, torso, head, or any portion of the body, including the whole body, can be exposed to the sound waves in a similar manner to what we described herein.

### Example 6. LFU Treatment of Aged Mice Improves Performance

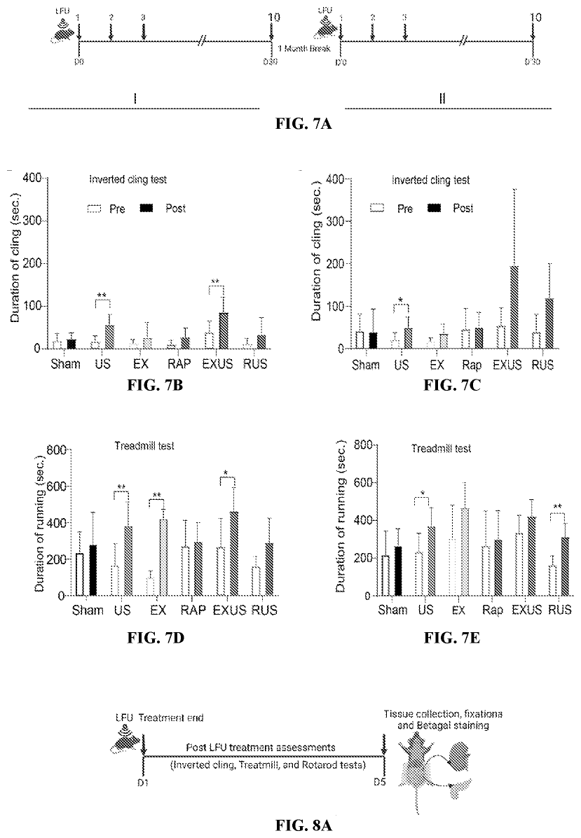

To determine if LFU therapy could benefit aged organisms, the inventors tested the effects of LFU therapy on aged mice by treating them for 30 minutes every 3 days. In these experiments, it was found that the physical performance of the LFU treated mice and particularly the LFU plus exercise treated mice were improved relative to the control group. The treatment plan is illustrated in FIG. 7A and shows that Mice were treated 30 min every 3^{rd} day for one month (all LFU groups) and exercise training sessions were conducted 3 times per week (all EX groups). Rapamycin was delivered to the mice through their food at a level that was approved by IACUC. Animals were treated a second time after a month break. Pre and post treatment performance assays were performed on each group (control (Sham), ultrasound (US), ultrasound plus exercise (EXUS), exercise (EX) rapamycin (Rap), and rapamycin plus ultrasound (RUS)) of eight mice (4 males and 4 females of 20-24 months in age of C57BL/6J strain) for each treatment period.

FIGS. 7A to 7E show the effects of ultrasound treatment on performance of aged mice. (FIG. 7A) Schematic illustration of the treatment plan. Mice (22-24 month old C57BL/6J strain) were LFU treated for 30 minutes every 3^{rd} day or ran on the treadmill 12 times for 20 minutes over one month. They then had a month break and were treated or exercised for a second month. Each study group contained four male and four female mice. (FIG. 7B) Graph of results of inverted cling test after the first month. and (FIG. 7C) after the second month of treatment. (FIG. 7D) Results of the treadmill test after the first month and (FIG. 7E) after the second month of treatment. Groups were untreated (Sham), treated with LFU (US), treated with exercise (EX), treated with rapamycin (RAP), treated with LFU plus exercise (EXUS); and treated with rapamycin plus LFU (RUS). Results are plotted as mean±S.D. Student t-test was used to determine the statistical significance. P-value greater than 0.05 is represented by ns. Statistical significance was given in p-value. *p value<0.05, **p value<0.001, and ***p value<0.0001.

### Example 7. LFU Rejuvenation of Cells in Mouse Tissues

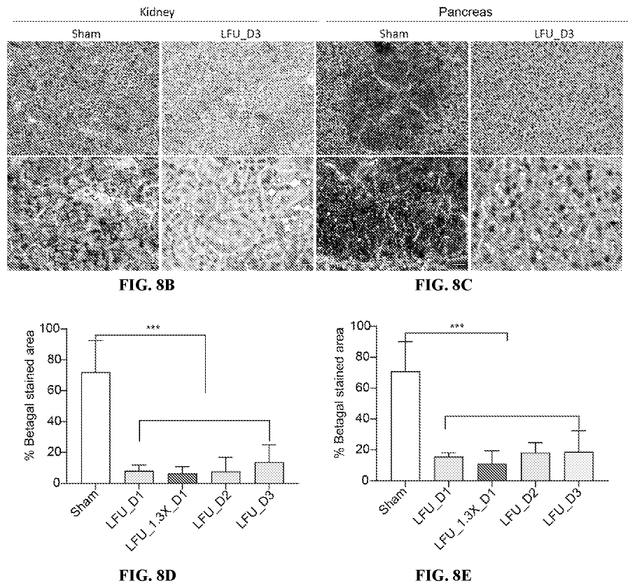

FIGS. 8A to 8E show that LFU decreases the fraction of senescent cells in kidney and pancreas. Groups of ten (5 males and 5 females) 22-25 months old mice were treated with LFU every day (LFU_D1), every other day (LFU_D2), every third day (LFU_D2) at 1× power plus every day at 1.3× power (LFU_1.3X_D1). After 2-4 weeks of treatment and assessment, mice were euthanized, and kidney and pancreas were collected for SA-β galactosidase staining. (FIG. 8A) SA-β galactosidase-stained kidney section of sham and LFU_D3 mice. The β galactosidase stain appear blue and is much more intense in the sections from the sham kidney. (FIG. 8B) SA-β galactosidase stained images of pancreas sections shown in color. Scale bar=150 μm. and 20 μm. (FIG. 8C) Quantification of β galactosidase-stained area of kidney section. β galactosidase staining was reduced significantly in all LFU treated mice. (FIG. 8D) Quantification of β galactosidase staining of pancreas sections. Results are plotted as mean±S.D. Student t-test was used to determine the statistical significance. P-value greater than 0.05 is represented by ns. Statistical significance was given in p-value. *p value<0.05, **p value<0.001, and ***p value<0.0001 (n=10 mice).

### Example 8 Ultrasound Enhancement of Wound Healing

In studies with skin wounds in mice, it was found that ultrasound can enhance the healing in young mice. An even greater relative enhancement of healing might well occur in the older animals through the rejuvenation of senescent cells. One application is the treatment of diabetic foot ulcers where generally older individuals having poor circulation due to diabetes and continue to have foot ulcers that do not heal (see FIG. 6).

Ultrasound coupling. The primary objective of the sound transduction was to create tensile stress in the membrane that is relatively constant for time periods on the order of one second while only causing acceptable friction at cell-cell adhesions thereby limiting adverse heat production. This is achieved with wavelengths much larger than a cell diameter. The constancy criterion requires the acceleration forces to be comparable to the resistance of pressure against the tissue, limiting the excitation to below 200 kHz. The wavelength at 200 kHz in tissue is some 7.5 mm, hence friction heating is generally not a limiting factor. Coupling low frequency sound waves into tissue without excessive reflections, requires a form of impedance matching. This is achieved using a contact material—which can be part of the transducer or the immersion itself—to be of a similar acoustic impedance to the tissue treated. Unlike the vast majority of ultrasound treatment methods, the method herein does not generally or at least not purposely aim to deposit energy in the specimen (unless explicitly otherwise stated in this text). Attenuation in the low frequency range is very low—it can be 20% for the transversal of the entire human body—and hence the energy delivered to any given volume of tissue is a minute fraction of the transmitted energy which is also the reason, why this text includes intricate detail on how to finally absorb the US waves after they transited the patient. This is fundamentally different from MHz range ultrasound that deposits virtually all its energy over the course of a few centimeters into the specimen. For most setups, and in particular the above listed ulcer and diabetic devices, the transmitted sound wave is actively uncoupled/destroyed after its passage through the target tissue. This can be achieved by impedance matched absorbers that have scattering bubbles in their interior. These special coupling and uncoupling requirements are unique properties of some embodiments of the devices that can be used with the present invention.

Ultrasound transducer. The inventors used ring transducers with diameters of 16 mm and 25 mm made of PZT4 and PZT8 material that can be effectively driven up to 100s of volts per millimeter thickness (Beijing Ultrasonics 25×10×4 and 16×8×4 piezoceramic rings). 33 The surface of those piezos (or piezo stacks) can produce large amplitudes of around up to 1.5 μm at low frequencies because they are thick and pre-tensioned and can hence contract and expand. An aluminum cone is mounted on the rings to widen the field to some 5 cm diameter and maintain the amplitude due to being a resonant structure, in order to form plane waves. Aluminum has good coupling to the PZT material but poorly matches the water bath due to its very different acoustic impedance. The inventors added epoxy and silicon rubberized coats to improve the emission and cap the internal reflections.

To avoid the near field of the transducer, a 2λ (2 wavelengths) deep 37° C. water bath was used between the top of the transducer to separate the specimen. The specimen itself was floated on the water surface inside a standard multi well plate (Thermo Scientific Nunclon Delta Surface 96 well plates). Those samples are tape sealed in order to prevent water creep under ultrasound irradiation. The well plates were suspended on a PET foil with a rectangular opening at the bottom on the plate. Polymer and glass cover slips both showed minimal attenuation of the beam strength.

To eliminate standing wave patterns that create spatial intensity fluctuations, the transducer base was suspended and mounted on absorber foam. The influence of surface reflections was minimized by sweeping the frequency 5% to 15%, leading to an about 80% homogeneity in the center of the sound cone. This finally allowed for controlling the power emitted via the driving voltage of the transducers. Both a resistor network and a class A amplifier were used with interchangeable results.

Despite the shielded wiring, radiated radio noise from the transducers remained considerable and the incubator cabinet was used as a shielding capsule. For open-air use shielded cables were used to feed the transducers.

Ultrasound generators. All primary signals and sequences were generated by ArduinoDue microcontrollers. This enabled altering duty cycles, sequences, and duration swiftly. The analog setup, used to calibrate the entire system, did output the waveform via the Arduino's digital-to-analog-converter, then amplified via a medium power amplifier (LM 675 from Texas Instruments) with a symmetric±24V supply. This drives a high frequency ferrite ring core (Richco Ferrite Ring Toroid Core, 31.5×19.3×8 mm) isolation 1:5 transformer feeding up to 200V amplitude to the transducers. Any controller and amplifier can be used as modern embedded controllers with a DAC can provide the output signals needed.

For higher power levels, the generator was operated as a switching power supply from a single 24V source. The 3.3V logic of the controller shuttered two transistor switches (Infineon IRFP260MPBF 50A, 200V N-Channel MOSFET) which drove each input of the same transformer. Pull-up was provided by 6, 8, or 10 Ohm power resistors with 50 W loss ratings. The transformer inputs were clamped by varistor (EPCOS Varistor 8 nF 20A 56V) for the switching design and by diodes (1N5408) to the supply power for the amplifier design. The tunable range was kept within 50% bandwidth of the design frequency of the transducer, i.e., a 30 kHz generator was used from 22.5 kHz to about 37.5 kHz. The controller outputs the waveform sample at 1 M sample/second. Exact 30 kHz can be output by storing 3 full waves in 100 samples which then are output 10000 times a second. A soft start and stop are provided by ramping up and down the amplitude over 30 waves, i.e., the same time-base provides accurate on-off cycles.

Calcium indicator dye-based assay. Cell samples were incubated with the calcium indicator dye (4 mM of Cal-520 AM, AAT Bioquest) for 1 hour. Samples were then replenished with fresh culture medium and allowed to stabilize for 30 minutes prior to ultrasound treatment.

Immunocytochemistry and fluorescence microscopy. Samples were fixed with 4% paraformaldehyde (Thermofisher Scientific) solution for 10 minutes and permeabilized with 0.2% Triton X-100 for 5 minutes. Normal goat serum (2%) was used as a blocking buffer and samples were treated with serum for 1 hour. Samples were then incubated with primary antibodies of rabbit polyclonal anti-Piezol (1:200, Novus Biologicals, catalogue no. NBP1-78446) overnight at 4° C. followed by treatment with Alexa Fluor-594 secondary antibodies (Thermofisher Scientific). Hoechst dye (1:1000, Thermofisher Scientific) was used to stain cell nuclei.

For the in vitro study, fluorescence and bright field images were acquired using wide-field Olympus live-EZ microscope equipped with a Photometrics CoolSNAP K4 camera and WI live-SR spinning disk microscope equipped with a Photometrics Prime 95B sCMOS camera. For in vivo tumor imaging, a wide-field Zeiss stereomicroscope was used.

Apoptosis and necrosis assay. To identify cell apoptosis, Annexin V-Alexa Fluor 488 or Annexin V-Alexa Fluor 594 conjugates (Thermofisher Scientific) were used according to the manufacturer's protocol. To check cell necrosis, propidium iodide from live/dead cell double staining kit (Sigma Aldrich) was used according to the manufacturer's protocol. Assay was performed at least 12 hours after the ultrasound treatment.

Live cell assay. Calcein AM (Sigma Aldrich) dye was used to check the cell viability according to manufacturer's protocol. Cells were incubated with dye every day for 15 minutes before imaging.

Cell line and cell culture. Human Foreskin Fibroblast (HFF) were purchased from the ATCC. African monkey kidney derived Vero cells were obtained from the M. Garcia-Blanco lab as a gift. All these cell lines were cultured as per manufacturer's protocol. Vero cells and HFF cells were in growth medium containing DMEM and 10% FBS. Cells plated at 20-40% confluency were maintained in an incubator at 37° C. and 5% CO₂.

Senescence induction and quantification. Vero cells were treated with various stressors including 200 μM H₂O₂, 4 μM of sodium butyrate (SB), and 25 μM of Bleomycin Sulphate (BS) and incubated for 36-48 hours. After washing with PBS and then replacing growth medium with fresh medium, cells were incubated for 4 days to confirm the growth arrest of senescent cells. Human foreskin fibroblast (HFF) cells were serially passaged up-to p15 since replication of these cells was dramatically reduced at p15-17. The inventors used four criteria to determine if cells were senescent; (1) Cell cycle arrest by determining the growth rate, (2) Increase in cell spread area, (3) Development of senescence associated secretory phonotype (SASP) in culture medium, and (4) beta-gal staining. The inventors captured images of cells with an Evos microscope at 10× magnification after treatment and 48 hours post treatment. To measure growth by the increase in cell number, 15 random images were captured, then the average number of cells were determined, which was divided by the area of one frame to get the cell density (cells/cm²). Then this seeding density was multiplied by the total area of the dish or well to obtain the total number of cells after US treatment and after 48 hours if incubation. The total number of cells at 48 hours was divided by the total number of cells just after the treatment to determine the growth rate. If the ratio was one, there was no growth.

Senescence was detected by the beta-gal senescence staining kit as per manufacturer's protocol. Briefly, sub confluent senescent cells were stained by the SA-beta gal staining solution and incubated overnight at 37° C. The Beta gal-stained cells appeared blue and were Senescent cells. The percentages of beta-gal positive cells were determined by counting the number of blue cells and dividing by the total number of cells. Cell spread area was determined by capturing the mages of cells with a 10× objective using an Evos microscope. Then, the inventors used ImageJ software to calculate spread area by manually encircling the cell periphery of each cell. The inventors used minimum 150 cells for the analysis. To determine the SASP activity, the inventors cultured the senescent cells for 3-4 days and then supernatant was collected from each dish. This supernatant was used to culture normal cells. Development of a senescence phenotype by the normal cells in supernatant medium confirmed that senescent cells were secreting SASP.

Ultrasound treatment of cells. Prior to ultrasound treatment, the plates containing senescent Vero cells or late passage HFF cells were wrapped with parafilm to avoid contamination and water influx into the plate. The samples were placed on the plastic mesh, which was mounted on the water tank with an ultrasound transducer. Water in the tank was degassed and heated to temperature of 35° C. The distance between the sample and transducer was approximately 9-10 cm. The inventors also ensured that there were no air-bubbles or air-water interfaces between the water and the sample. Output power of transducer was measured at the plate location by hydrophone. Cells were treated with pressure pulses of 3.5-4.0 pa. using 32.249 kHz frequency ultrasound for 30 minutes. Cells were treated with 1.5 seconds on and 1.5 seconds off cycle. After ultrasound treatment, cell plates were returned to the incubator for 48 hours to determine the growth of senescent cells.

Reversal of senescence. First, the inventors induced senescence in Vero cells using sodium butyrate, then the inventors confirmed senescence using growth arrest and Beta gal staining methods. The inventors treated the senescent cells using ultrasound of optimized parameters (33 kHz frequency and 3.5-4 Pa). The inventors incubated these cells for 48 hours and measured the growth and morphology of the cells before the trypsinization. The inventors named this passage P0. These cells were then trypsinized, reseeded, and incubated for 48 hours in the P1 passage. This process was repeated to P3 passage. The inventors evaluated the cells in terms of fold change in number for growth, morphology, beta-gal, and EDU staining to check the population of senescent cells. Senescent cells without ultrasound treatment were used as control cells. Typically, by passage P3, the senescent cells exhibited the phenotype of normal proliferating cells.

Passage 15-24 HFF cells were treated with ultrasound at the optimized frequency and power. Cell proliferation was determined by counting the number of cells at the time of seeding and 48 hours post US treatment. Ultrasound treated HFF cells showed higher fold change in growth than the untreated HFF cells. In the case of P24 HFF cells, they were treated with ultrasound and incubated for 96 hours prior to trypsinization, reseeding and incubation for 48 hours. Proliferation and morphology were measured after 48 of incubation. P24, US treated HFF cells became smaller in size, and they also showed dramatically greater proliferation than the untreated P24 HFF cells.

Ultrasound treatment of Aged mice. Aged mice (21-24 months old) were treated in a big 4L glass beaker with one plastic cylinder of 13 cm height and 152 cm in diameter. A metal mesh was placed on top the cylinder that supported the mice and enabled them to rest their four limbs and body in the water. Degassed and 32-35° C. warm water was poured into the beaker. Level of water kept 1 inch above the metal mesh so that the half of the body of the mice remained in water. Once the mice were placed in the water, intermittent ultrasound of 32.249 kHz and 3.5-4 Pa was applied to the mice for 30′ at 1.5 second on and 1.5 second off. Animals in the ultrasound groups were treated at 72-96 hour intervals for one month (10 treatments). During the ultrasound treatment, mouse activity was observed and their adaptation to the system. After treatment, the animals were placed in separate cage with tissue paper to dry the animals and then they were returned to their home cage. Control mice were placed in the same water bath for 30′ without ultrasonication. In the ultrasound treatment procedure, Animals were in direct contact with the water. The reason for putting animals in water was that ultrasound is attenuated dramatically at air-water interfaces.

Physical assessment of the mice. For assessment of the effect of ultrasound (US) treatment on physical performance of the mice, the inventors used 6 groups of old mice, 1. Sham 2. Ultrasound treatment. 3. Exercise 4. Rapamycin 5. Exercise plus Ultrasound 6. Ultrasound treatment plus Rapamycin. Each group comprised of four males and four females. In the case of the rapamycin-treated animals, the C57BL/6J mice were fed with encapsulated rapamycin and monitored daily for a month. Animals of ultrasound groups were treated every 72-96 hours for a month. Animals in the exercise group were trained three times per week on a treadmill for 25′ in each exercise training session. Prior to starting the experiment, the physical functions and health condition of the mice were assessed, referred as pre-assessment. After one month of ultrasound treatment and exercise sessions, the physical performance and health conditions of the animals were again assessed, referred as post-assessment. Physical performance was determined by the functional assessment tests including Grip test, Rotarod, Treadmill, and Inverted Cling tests.

Treadmill. The mice were tested for the maximum power output/maximum gait speed and endurance (to exhaustion) by running on a treadmill. The outcome measurement was the running duration. During the training session, the mice were familiarized with the device, first running at a constant speed, later increasing the speed progressively one unit in every 20 seconds. The mice were allowed to rest 10 minutes between trials. Three electric shocks of 0.4 mA ended the trial and animals were given three trials. During the test session, the speed was increased, and the mouse was allowed to run as long as they could before getting three shocks

Inverted Cling: This grip test was useful to quantify muscle strength and endurance by measuring how long the mouse held onto the grid while inverted. Each animal was tested 2 times with a resting break of 10 minutes between trials. A minimum of 10 seconds holding was required for test validation to exclude a slip. Three trials were conducted in the first test and then two trials after a gap of 10 minutes.

Exercise training sessions. Animals of Exercise and Exercise+ ultrasound groups were exercised on a treadmill for 25 minutes in every 48 hours, three times in a week for one month. Every exercise session was preceded by 10 minute warm up at 6 cm/s, followed by 10 minute training at 8 cm/s, and 5 minute cool down at 6 cm/s. Training was progressive starting at 8 cm/s in the first week and increasing to 11 cm/s in week 4. The mice were encouraged to run on treadmill with a light electric shock when they stopped running.

There were 12 exercise training sessions and 10 ultrasound treatments in the one-month experimental period.

Mitochondrial morphology. Ultrasound treated cells were incubated with Mitotracker (Invitrogen) in 100 Um at 37° C. for 30 minutes. Then, Images were captured in Confocal microscope at 15 randomized fields per sample for quantification. AR and form factors are determined using the formula Major axis/Minor axis and Perimeter2/(4π×surface area)1.

Immunofluorescence staining. Cells were seeded on 27 mm glass bottom dish (Ibidi), after ultrasound irradiation, cells were washed twice with PBS. Then fixed with 4% paraformaldehyde for 10 minutes and permeabilized with 0.2% Triton-X-100 for 5 minutes. The cells were washed with PBS thrice and incubated with 3% blocking buffer for 1 hour. Anti-LAMP primary antibody (1:400) was incubated with sample at 4° C. overnight followed by washing and incubation with Alexa flour 488 secondary antibody (1:1000) at room temperature for 4 hours. Images were captured by confocal microscope.

Mitochondrial ROS. MitoSOX (Invitrogen) red was used to measure the mitochondrial reactive oxygen species production. Briefly, 5 ul of MitoSOX was added to the growth medium for 10 minutes at 37° C. Fluorenece images were captured in confocal microscope (Olympus). ROS level was determined by the intensity measurement.

Live and dead cell assay. Live cells were detected using Calcine AM (Sigma Aldrich) as per manufacturer's instructions. Briefly, adherent cells were treated with Calcein AM in 2000:1 in Opti-MEM medium and incubated for 30 minutes. Apoptotic/Dead cells were identified using Annexin V-FITC/Propidium iodide (PI) (Sigma Aldrich)) as per the manufacturer's instructions. Live and dead assay was performed immediately after the ultrasound irradiation and post 24 hours of treatment.

Calcium release assay. Release of calcium was measured using calcium dye (4 mM of Cal-520 AM, AAT Bioquest) as per manufacturer's protocol. Semiconfluent cells were incubated in calcium dye for 30 minutes prior to ultrasound irradiation.

Statistical analysis. All results are shown as mean±s. d. and paired t-test, two tails used for two groups. GraphPad Prism 8.4.3. was used for making graph and statistical analysis. * P values<0.05, **p values<0.002, ***p values<0.001, and non-significant (ns) p-value>0.05 were used, shown in figures.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Gorgoulis, V. et al. Cellular Senescence: Defining a Path Forward. Cell 179, 813-827 (2019).
2. Krimpenfort, P. & Berns, A. Rejuvenation by Therapeutic Elimination of Senescent Cells. Cell 169, 3-5 (2017).
3. Neves, J., Sousa-Victor, P. & Jasper, H. Rejuvenating Strategies for Stem Cell-based Therapies in Aging. Cell Stem Cell 20, 161-175 (2017).
4. Blood factors transfer beneficial effects of exercise on neurogenesis and cognition to the aged brain|Science. https://science.sciencemag.org/content/369/6500/167.
5. Hubackova, S. et al. Selective elimination of senescent cells by mitochondrial targeting is regulated by ANT2. Cell Death Differ. 26, 276-290 (2019).
6. Baar, M. P. et al. Targeted Apoptosis of Senescent Cells Restores Tissue Homeostasis in Response to Chemotoxicity and Aging. Cell 169, 132-147.e16 (2017).
7. Kirkland, J. L. & Tchkonia, T. Cellular Senescence: A Translational Perspective. EBioMedicine 21, 21-28 (2017).
8. Cai, Y. et al. Elimination of senescent cells by β-galactosidase-targeted prodrug attenuates inflammation and restores physical function in aged mice. Cell Res. (2020) doi:10.1038/s41422-020-0314-9.
9. Libertini, G., Ferrara, N., Rengo, G. & Corbi, G. Elimination of Senescent Cells: Prospects According to the Subtelomere-Telomere Theory. Biochem. Mosc. 83, 1477-1488 (2018).

1. A non-invasive method of treating aging comprising:
mechanically stretching at least one living cell in an amount sufficient to delay at least one aging characteristic.

2. The method of claim 1, wherein the at least one aging characteristic is selected from reduced cellular senescence, increased cell division, reduced cell size, increase secretion of growth factors, decreased secretion of senescent factors, prevented mitochondrial fusion, increased mitochondrial fission, or enhanced wound healing.

3. The method of claim 1, wherein at least one of:
mechanically stretching the at least one living cell is by applying a repetitive low frequency ultrasound that is configured to target a region in need of treatment for aging, wherein the region is targeted by one or more ultrasound sources pointed at the target from one or more directions;
mechanically stretching the at least one living cell is by applying a repetitive low frequency ultrasound treatment is selected from at least one of: standing wave patterns, shock waves, rapid sounds transitions, square waves, sawtooth waves, random waves, or undulating a low-high intensity beat;
mechanically stretching the at least one living cell is by applying a repetitive low frequency ultrasound treatment is between about 5 minutes to about 60 minutes in duration and is repeated for a period selected from the group consisting of: once a day, about once every two days, about once every three days, about once a week, and about twice a week;
mechanically stretching the at least one living cell is a sequence of programmed cycles of waves generated according to a treatment plan or a type of therapeutic procedure, a treatment plan is generated using one or more machine learning techniques, a lower frequency modulation of a sound wave, a cyclical on-off pattern with a set duty-cycle, a variation of amplitude, frequency, and phase; a superposition of several carrier frequencies each variable in phase and center frequency and relative intensity; modulated by an external input signal, modulated by an audible sound signal; or
mechanically stretching the at least one living cell is a repetitive low frequency ultrasound treatment delivered to at least a localized region of a body of a subject; or
wherein at least a portion of mechanically stretching the at least one living cell is a repetitive low frequency ultrasound treatment applied concurrently with one or more active agents that reduce cellular senescence.

4. The method of claim 3, wherein an immersion design for the treatment is optimized to treat wounds and ulcers and can include one or more chambers that are generally shallow and suited for full immersion with an absorber shield about a treatment zone, and wherein the one or more one or more ultrasound sources and an absorber can contact a patient or are insulated by an immersion medium.

5.-11. (canceled)

12. The method of claim 3, wherein the active agent is a supernatant of cells treated with a repetitive low frequency ultrasound treatment.

13.-14. (canceled)

15. The method of claim 1, further comprising providing a controller, wherein the controller is: configured to control one or more ultrasound transducers based on sensor data; integrated with one or more ultrasound generators; comprises remote drivers that limit energy from the one or more ultrasound generators to provide patient safety; neutral drivers with a voltage across two or more ultrasound transducer elements that amounts to zero at all times; or connected to one or more transducer stacks that reduces a peak voltage used by one or more ultrasound generators.

16.-17. (canceled)

18. The method of claim 3, wherein a repetitive low frequency ultrasound treatment is provided by a piezo transducer, a voice coil, a capacitive membrane, fluidic instability, a shuttered hydraulic, a pneumatic device, a spark discharge, chemically generated pressure waves, engine driven sound, induced muscle tonus, a transducer array, a phased array with a synthetic aperture, or harmonics of the frequencies.

19. The method of claim 1, wherein at least one of:
the at least one living cell is a plurality of living cells and applying a repetitive low frequency ultrasound treatment extends a replicative lifespan of the at least one living cell;
the at least one living cell is a cell line, or in a tissue, organ, limb, or whole body;
the at least one living cell is a plurality of living cells, and the method further comprises applying repetitive low frequency ultrasound treatment to a plurality of in vivo living cells; or
the at least one living cell is a plurality of living cells and applying a repetitive low frequency ultrasound treatment that reverts a plurality of living cells to a more youthful phenotype.

20. (canceled)

21. The method of claim 1, further comprising providing a mechanism that at least one of: absorbs or uncouples a repetitive low frequency ultrasound treatment to reduce or deflect a sound wave after patient treatment, is a material that is suitable to at least one of attenuate sound waves or guide the sound waves away from a site or treatment, or includes one or more elements that convert residual sound energy into heat.

22. A method of reducing cellular senescence comprising:
applying a repetitive low frequency ultrasound treatment to at least one living cell, wherein the repetitive low frequency ultrasound treatment delays at least one aging characteristic.

23. The method of claim 22, wherein a wavelength of the low frequency ultrasound treatment is equal to or greater than an average cell diameter.

24. The method of claim 22, wherein the at least one aging characteristic is selected from reduced cellular senescence, increased cell division, reduced cell size, increased secretion of growth factors, decreased secretion of senescent factors, prevented mitochondrial fusion, increased mitochondrial fission, or enhanced wound healing.

25. The method of claim 22, wherein at least one of:
applying the repetitive low frequency ultrasound is configured to target a region in need of treatment for aging, wherein the region is targeted by one or more ultrasound sources pointed at the target region from one or more directions;
applying the repetitive low frequency ultrasound treatment is selected from at least one of: standing wave patterns, shock waves, rapid transitions, square waves, sawtooth waves, random waves, or undulating a low-high intensity beat;
applying the repetitive low frequency ultrasound treatment is between about 5 minutes to about 30 minutes in duration and is repeated for a period selected from the group consisting of: once a day, about once every two days, about once every three days, about once a week, and about twice a week;
applying the repetitive low frequency ultrasound treatment is for 1, 2, 3, 4, 5, 6, 7, 8, 9, 10, 15, 20, 25, 30, 40, 50, 60, 70, 75, 80, 90 minutes, or 2, 3, 4, 5, 6, 7, 8, 9, 10, 11, 12, or 24 hours;
applying the repetitive low frequency ultrasound treatment is at 5, 10, 15, 20, 25, 30, 35, 40, 45, 50, 60, 75, 100, 150, 200, 250, 300, 400, 500, 600, 700, 750, 800, 900 kHz, 1 MHz or higher; or
wherein at least a portion of an application of the repetitive low frequency ultrasound treatment is applied concurrently with an active agent that reduces cellular senescence.

26.-29. (canceled)

30. The method of claim 22, wherein applying the repetitive low frequency ultrasound treatment is at 10, 20, 50, 100, 150, 200, 250, 300, 350, 400, 450, 500, 1,000, 5,000, 10,000, or <500 mW/cm².

31.-32. (canceled)

33. The method of claim 25, wherein the active agent is a supernatant of the at least one living cells treated with the repetitive low frequency ultrasound treatment.

34. The method of claim 22, wherein at least one of:
the repetitive low frequency ultrasound treatment is applied to the at least one living cell until the at least one living cell reaches cellular senescence to delay at least one aging characteristic;
the repetitive low frequency ultrasound treatment is delivered from at least one of:
one or more ultrasound transducers configured to generate a sequence of programmed cycles of waves;
one or more ultrasound transducers mounted to a robotic arm, wherein the robotic arm is controlled to position the one or more ultrasound transducers into a desired location or orientation;
one or more ultrasound transducers are phased array ultrasound transducers; or
the one or more ultrasound transducers are sealed and water-proof;
wherein a frequency, a magnitude of a periodic force and a period of time are determined based at least in part on a type of target cells or based on the output of a feedback sensor at or near a treatment zone; or
wherein a wavelengths of a wave is in the order of a size of an organ and an amplitude in the order of single cells; or
the repetitive low frequency ultrasound treatment is at least one of: a sequence of programmed cycles of waves generated according to a treatment plan or a type of therapeutic procedure, a treatment plan is generated using one or more machine learning techniques, a lower frequency modulation of a sound wave, a cyclical on-off pattern with a set duty-cycle, a variation of amplitude, frequency, and phase; a superposition of several carrier frequencies each variable in phase and center frequency and relative intensity; modulated by an external input signal, modulated by an audible sound signal;
the repetitive low frequency ultrasound treatment is delivered to at least a localized region of a body of a patient;
the repetitive low frequency ultrasound treatment is provided by a piezo transducer, a voice coil, a capacitive membrane, fluidic instability, a shuttered hydraulic, a pneumatic device, a spark discharge, chemically generated pressure waves, engine driven sound, induced muscle tonus, a phased array, a phased array with one or more synthetic apertures, or harmonics of the frequencies.

35.-36. (canceled)

37. The method of claim 22, further comprising providing a controller or controllers, wherein the controller is: configured to control one or more ultrasound transducers based on sensor data; integrated with one or more ultrasound generators; comprises remote drivers that limit an energy from the one or more ultrasound generators to provide patient safety; neutral drivers with a voltage across two or more ultrasound transducer elements that amounts to zero at all times; or connected to one or more transducer stacks that reduces a peak voltage used by one or more ultrasound generators.

38. The method of claim 22, wherein at least one of:
the at least one living cell is a plurality of living cells, and the method further comprises applying repetitive low frequency ultrasound treatment to a plurality of in vivo living cells;
the at least one living cell is a cell line, or in a tissue, organ, limb, or whole body;
the at least one living cell is a plurality of living cells and applying the repetitive low frequency ultrasound treatment extends a replicative lifespan of the at least one living cell; or
the at least one living cell is a plurality of living cells and applying the repetitive low frequency ultrasound treatment reverts a plurality of living cells to a more youthful phenotype.

39.-42. (canceled)

43. The method of claim 22, further comprising providing a mechanism that at least one of: absorbs or uncouples the repetitive low frequency ultrasound treatment to reduce or deflect a sound wave after patient treatment, is a material that is suitable to at least one of attenuate the sound waves or guide the sound waves away from a site or treatment, or includes one or more elements that convert residual sound energy into heat or providing an immersion vessel to treat wounds and ulcers that includes one or more chambers that are partial or full immersion with an absorber shield about a treatment zone, and wherein one or more transducers and one or more absorber can contact a patient or are insulated by an immersion medium.

44. (canceled)
